(19) United States
(12) Patent Application Publication
(10) Pub. No.: US 2026/0076919 A1
(43) Pub. Date: Mar. 19, 2026

(54) LAYER-BY-LAYER DELIVERY OF ACTIVE AGENTS

(71) Applicant: Luna Labs USA, LLC, Charlottesville, VA (US)

(72) Inventors: Yang XU, Charlottesville, VA (US); Ziyou ZHOU, Charlottesville, VA (US); Brad BROOKS, Charlottesville, VA (US); Christopher K. TISON, Charlottesville, VA (US)

(21) Appl. No.: 19/109,171

(22) PCT Filed: Jul. 6, 2023
(86) PCT No.: PCT/US2023/069681

§ 371 Date: Mar. 6, 2025

Related U.S. Application Data
(60) Provisional application No. 63/404,701, filed on Sep. 8, 2022.

(51) Int. Cl. A61K 9/51; A61K 39/00; A61K 39/015; A61K 39/21; A61P 37/04

(52) U.S. Cl. CPC A61K 9/5161; A61K 9/5192; A61K 39/015; A61K 39/21; A61P 37/04; A61K 2039/53; A61K 2039/55555

(57) ABSTRACT

Provided herein are pharmaceutical compositions comprising chitosan based nanoparticles, such as those having a core-shell structure, which can be configured for layer-by-layer delivery of active agent(s).

### CROSS-REFERENCE TO RELATED APPLICATIONS

This application claims the benefit of U.S. Provisional Application No. 63/404,701, filed Sep. 8, 2022, the content of which is incorporated herein by reference in its entirety.

### GOVERNMENT'S INTERESTS

The invention was made with government support under the following contract numbers: Contract No. W81XWH-18-C-0073 awarded by the US Army; and W81XWH-21-C-0057 awarded by DHA. The U.S. government has certain rights in the invention.

### BACKGROUND

### Field of the Disclosure

In various embodiments, the present disclosure generally relates to nanoparticulate formulations for delivering active agents, such as proteins, antigens, vaccines, adjuvants, etc.

### Background

Pathogen transmission is a widespread threat to global human health. Vaccines are critical during pandemic outbreaks, and vaccination is considered one of the most successful, affordable, and sustainable methods for prevention and elimination of infectious diseases. Many of the infectious diseases that have historically caused widespread morbidity and mortality in young children have been largely controlled by effective vaccines. Previously common diseases such as diphtheria, tetanus, poliomyelitis, smallpox and measles can be prevented using relatively simple vaccines that stimulate robust antibody responses. Notable exceptions include malaria, tuberculosis, and human immunodeficiency virus (HIV) infection. These pathogens have sophisticated mechanisms to evade human immune responses and cannot be prevented effectively by antibodies alone. (Lee, et al. 2020.) Thus, the first challenge for development of effective vaccines relates to the characteristics of the pathogens themselves. Effective delivery systems or adjuvants are required for these disease vaccines to optimize the immune responses and improve the effectiveness of vaccines.

Currently, novel vaccine delivery systems or adjuvants are required to be more rationally constructed or selected to direct the immune system toward an effective response. The combined adjuvant features of dose reduction and antigen sparing can have important implications for improving global vaccine supply. In addition, delivery systems or adjuvants can improve immune responses in populations where responses to vaccines are typically reduced, such as infants, the elderly, and the immunocompromised (Genç, et al. 2020). For the past several decades, very few delivery systems or adjuvants have been approved for human use by the US Food and Drug Administration (FDA), except aluminum salts, MF59, virosomes, montanide ISA 51, AS01, CpG 1018, liposome, or lipid nanoparticles. This lack of approved adjuvants is despite significant study of many different materials for potential use. To further improve vaccine formulations with better adjuvants, a considerable amount of effort has been dedicated to discovering alternatives, including synthetic polymeric nanoparticles. Many studies have been conducted to develop biocompatible and biodegradable polymer nanoparticles to replace conventional adjuvants. The ability of biopolymers to act as adjuvants depends largely on their extrinsic and intrinsic properties including polymer structure, amphiphilicity, and surface charge display of self-assembled structures (Shakya and Nandakumar 2012). Polymeric delivery systems or adjuvants can complex with antigens easily via physical or chemical interactions. The facile manipulation of polymer chemistry will enable the adjuvants to be tailored for the antigen of interest. It can also protect antigens from degradation, increase antigen stability and provide slow release, and enhance the antigen presenting cell uptake and activation, thereby enhancing immunogenicity at reduced doses (Yan, et al. 2020), etc.

In recent years, nanotechnology has played an important role in the development of novel vaccine adjuvants or nano-delivery systems. The crucial parameters in vaccination are the generation of memory response and protection against infection, while an important aspect is the effective delivery of antigen in an intelligent manner to evoke a robust immune response. In this regard, nanotechnology is contributing to developing efficient vaccine adjuvants. Nanoparticles made from biocompatible and natural polymers such as chitosan, alginate, hyaluronic acid, gums, and β-glucan in a nanomaterial form have shown great potential as adjuvants or delivery systems for vaccine formulation. (Lee, et al. 2020.) Chitosan, particularly chitosan salts, have now been used in several preclinical and clinical studies with good tolerability, excellent immune stimulation, and positive clinical results across a number of infections (Watts and Smith 2014). As an example, ChiSys® has been used as a nasal vaccine delivery platform (in some instances combined with other adjuvants) for several different antigens, including diphtheria, seasonal influenza, avian influenza, and anthrax. Both enhanced antibody responses and efficacy have been demonstrated (Jabbal-Gill 2010).

According to the antiviral performance and immunomodulation of these chitosan-based biopolymers (S. Bashiri 2020), they will play significant and unique role in vaccine formulation for diseases such as malaria, AIDS/HIV infection, tuberculosis, influenza, COVID-19, etc.

### BRIEF SUMMARY

The present disclosure is based, in part, on the discovery that crosslinked cationic chitosan (in particular, N-trimethyl chitosan) nanoparticles can be used to provide an adjuvant effect and can be used for preparing formulations having multiple payloads which allow layer-by-layer delivery. As shown in the Examples section, these modified chitosan based nanoparticles are safe. Further, the Examples section shows an in vivo proof of concept delivery of multiple antigens for malaria which induced potent and long-lasting effect. The chitosan-based adjuvants can also elicit innate immune response that was potent to mediate non-specific anti-malarial effect.

In various embodiments, the present disclosure provides pharmaceutical compositions comprising crosslinked cationic chitosan nanoparticles, which typically have a core-shell structure. In some embodiments, the present disclosure also provides modified release formulations comprising drug-loaded nanoparticles based on crosslinked cationic chitosan, which can be configured to allow layer-by-layer delivery of various payloads. Methods of preparing the pharmaceutical compositions and modified release formulations are also described herein. In some embodiments, the present disclosure further provides method of using the pharmaceutical compositions and modified release formulations for stabilizing active agents and for delivering active agents to a subject in need to treat or prevent a disease or disorder described herein.

The present disclosure provides the following numbered exemplary embodiments 1-54:
Embodiment 1. A pharmaceutical composition comprising nanoparticles having a core-shell structure, wherein the nanoparticles comprise a crosslinked polymer comprising a cationic chitosan and an anionic cross-linker, wherein the nanoparticles have an average particle size of about 40 nm to about 1 μm as determined by Dynamic Light Scattering.
Embodiment 2. The pharmaceutical composition of Embodiment 1, wherein the cationic chitosan comprises quaternized ammonium cations.
Embodiment 3. The pharmaceutical composition of Embodiment 1 or 2, wherein the cationic chitosan is water soluble at a neutral pH, preferably, the cationic chitosan has an aqueous solubility at least 10 mg/ml at pH 5-8.
Embodiment 4. The pharmaceutical composition of any of Embodiments 1-3, wherein the cationic chitosan is N-trimethylated chitosan, with a degree of quaternization of between about 20% to about 60%, as determined by ¹H-NMR.
Embodiment 5. The pharmaceutical composition of any of Embodiments 1-4, wherein the cationic chitosan is prepared by treating a chitosan with a methylating agent (e.g., MeI), wherein the chitosan is characterized as having a degree of deacetylation of 75-85% and an average viscosity molecular weight (Mᵥ) of about 50,000-190,000 Daltons.
Embodiment 6. The pharmaceutical composition of any of Embodiments 1-5, wherein the anionic cross-linker is tripolyphosphate.
Embodiment 7. The pharmaceutical composition of any of Embodiments 1-6, wherein the nanoparticles comprise N-trimethylated chitosan and tripolyphosphate, with a weight ratio of N-trimethylated chitosan to tripolyphosphate ranging from about 2:1 to about 20:1, preferably, about 5:1 to about 10:1, more preferably, about 5:1 to about 7:1.
Embodiment 8. The pharmaceutical composition of any of Embodiments 1-7, wherein the nanoparticles further comprise a surfactant, such as a non-ionic surfactant, e.g., Tween™ 80.
Embodiment 9. The pharmaceutical composition of any of Embodiments 1-8, wherein the nanoparticles have an average particle size of about 40 nm to about 500 nm, or 150 nm to about 500 nm, preferably, about 200 nm to about 400 nm, as determined by Dynamic Light Scattering.
Embodiment 10. The pharmaceutical composition of any of Embodiments 1-9, further comprising at least one active agent, which is encapsulated within the nanoparticles and/or adsorbed on the surface of the nanoparticles.
Embodiment 11. The pharmaceutical composition of Embodiment 10, wherein the at least one active agent is a small molecule drug, a protein, a nucleic acid, a vaccine, or a therapeutic agent, or an adjuvant, preferably, the active agent is negatively charged (PI<7) at pH 7 or higher, or the active agent is a hydrophobic molecule, such as a small molecule drug having a Log P of at least 1, e.g., 1-5.
Embodiment 12. The pharmaceutical composition of any of Embodiments 1-11, wherein the nanoparticles further comprise a coating layer.
Embodiment 13. The pharmaceutical composition of Embodiment 12, wherein the coating layer comprises a negatively charged biocompatible polymer.
Embodiment 14. The pharmaceutical composition of Embodiment 13, wherein the coating layer comprises hyaluronic acid salt (e.g., sodium hyaluronate) or polystyrene sulfonate (e.g., sodium polystyrene sulfonate).
Embodiment 15. The pharmaceutical composition of any of Embodiments 1-14, wherein the nanoparticles have a zeta potential ranging from about −40 mV to about 50 mV.
Embodiment 16. The pharmaceutical composition of any of Embodiments 12-15, wherein the coating layer is present in an amount such that the weight ratio of the cationic chitosan (e.g., N-trimethylated chitosan) to the coating layer is in the range of about 1:1 to about 200:1, such as about 5:1 to about 20:1.
Embodiment 17. The pharmaceutical composition of any of Embodiments 12-15, wherein (1) the nanoparticles comprise N-trimethylated chitosan and tripolyphosphate, with a weight ratio of N-trimethylated chitosan to tripolyphosphate ranging from about 2:1 to about 20:1, preferably, about 5:1 to about 10:1, more preferably, about 5:1 to about 7:1; and (2) the coating layer comprises polystyrene sulfonate (e.g., sodium polystyrene sulfonate), and the weight ratio of N-trimethylated chitosan to polystyrene sulfonate ranges from about 1:1 to about 200:1, preferably about 5:1 to about 50:1, such as about 10:1 or 20:1.
Embodiment 18. The pharmaceutical composition of any of Embodiments 12-15, wherein (1) the nanoparticles comprise N-trimethylated chitosan and tripolyphosphate, with a weight ratio of N-trimethylated chitosan to tripolyphosphate ranging from about 2:1 to about 20:1, preferably, about 5:1 to about 10:1, more preferably, about 5:1 to about 7:1; and (2) the coating layer comprises hyaluronic acid salt (e.g., sodium hyaluronate), and the weight ratio of N-trimethylated chitosan to hyaluronic acid salt ranges from about 1:1 to about 200:1, preferably about 5:1 to about 50:1, such as about 10:1 or 20:1.
Embodiment 19. A modified release formulation comprising:
   (1) drug-loaded nanoparticles having a core-shell structure, wherein the drug-loaded nanoparticles comprise a crosslinked polymer comprising a cationic chitosan and an anionic cross-linker, a first active agent, and a second active agent; and
   (2) a layer coating the drug-loaded nanoparticles;
   wherein the first active agent is encapsulated within the drug-loaded nanoparticles and the second active agent is adsorbed on the surface of the drug-loaded nanoparticles, wherein the first and second active agents can be the same or different active agents, and wherein the drug-loaded nanoparticles have an average particle size of about 40 nm to about 1 μm as determined by Dynamic Light Scattering.
Embodiment 20. The modified release formulation of Embodiment 19, wherein the cationic chitosan comprises quaternized ammonium cations.
Embodiment 21. The modified release formulation of Embodiment 19 or 20, wherein the cationic chitosan is water soluble at a neutral pH, preferably, the cationic chitosan has an aqueous solubility of at least 10 mg/ml at pH 5-8.
Embodiment 22. The modified release formulation of any of Embodiments 19-21, wherein the cationic chitosan is N-trimethylated chitosan, with a degree of quaternization of between about 20% to about 60%, as determined by ¹H-NMR.
Embodiment 23. The modified release formulation of any of Embodiments 19-22, wherein the cationic chitosan is prepared by treating a chitosan with a methylating agent (e.g., MeI), wherein the chitosan is characterized as having a degree of deacetylation of 75-85% and an average viscosity molecular weight (Mᵥ) of about 50,000-190,000 Daltons.
Embodiment 24. The modified release formulation of any of Embodiments 19-23, wherein the anionic cross-linker is tripolyphosphate.
Embodiment 25. The modified release formulation of any of Embodiments 19-24, wherein the drug-loaded nanoparticles comprise N-trimethylated chitosan and tripolyphosphate, with a weight ratio of N-trimethylated chitosan to tripolyphosphate ranging from about 2:1 to about 20:1, preferably, about 5:1 to about 10:1, more preferably, about 5:1 to about 7:1.
Embodiment 26. The modified release formulation of any of Embodiments 19-25, wherein the first and second active agents are independently a small molecule drug, a biologic, a protein, a peptide, a nucleic acid, a vaccine or a therapeutic agent, or an adjuvant, preferably, the first and/or second active agents are negatively charged (PI<7) at pH 7 or higher, or the first and/or second active agents are hydrophobic molecules such as small molecule drugs having a Log P of at least 1, e.g., 1-5.
Embodiment 27. The modified release formulation of any of Embodiments 19-26, wherein the drug-loaded nanoparticles comprise the first and second active agents in a total amount of about 10-100% by weight of the cationic chitosan.
Embodiment 28. The modified release formulation of any of Embodiments 19-27, wherein the coating layer comprises a negatively charged biocompatible polymer.
Embodiment 29. The modified release formulation of any of Embodiments 19-27, wherein the coating layer comprises hyaluronic acid salt (e.g., sodium hyaluronate) or polystyrene sulfonate (e.g., sodium polystyrene sulfonate).
Embodiment 30. The modified release formulation of any of Embodiments 19-27, wherein the coating layer is present in an amount such that the weight ratio of the cationic chitosan (e.g., N-trimethylated chitosan) to the coating layer is in the range of about 1:1 to about 200:1, such as about 5:1 to about 20:1.
Embodiment 31. The modified release formulation of any of Embodiments 19-27, wherein (1) the drug-loaded nanoparticles comprise N-trimethylated chitosan and tripolyphosphate, with a weight ratio of N-trimethylated chitosan to tripolyphosphate ranging from about 2:1 to about 20:1, preferably, about 5:1 to about 10:1, more preferably, about 5:1 to about 7:1; and (2) the coating layer comprises polystyrene sulfonate (e.g., sodium polystyrene sulfonate), and the weight ratio of N-trimethylated chitosan to polystyrene sulfonate ranges from about 1:1 to about 200:1, preferably about 5:1 to about 50:1, such as about 10:1 or 20:1.
Embodiment 32. The modified release formulation of any of Embodiments 19-27, wherein (1) the drug-loaded nanoparticles comprise N-trimethylated chitosan and tripolyphosphate, with a weight ratio of N-trimethylated chitosan to tripolyphosphate ranging from about 2:1 to about 20:1, preferably, about 5:1 to about 10:1, more preferably, about 5:1 to about 7:1; and (2) the coating layer comprises hyaluronic acid salt (e.g., sodium hyaluronate), and the weight ratio of N-trimethylated chitosan to hyaluronic acid salt ranges from about 1:1 to about 200:1, preferably about 5:1 to about 50:1, such as about 10:1 or 20:1.
Embodiment 33. The modified release formulation of any of Embodiments 19-32, wherein the drug-loaded nanoparticles further comprise a surfactant, such as a non-ionic surfactant, e.g., Tween™ 80.
Embodiment 34. The modified release formulation of any of Embodiments 19-33, wherein the drug-loaded nanoparticles have an average particle size of about 40 nm to about 600 nm, or about 150 nm to about 500 nm, preferably, about 200 nm to about 400 nm, as determined by Dynamic Light Scattering.
Embodiment 35. The modified release formulation of any of Embodiments 19-34, wherein the coated drug-loaded nanoparticles have a zeta potential ranging from about −40 mV to about 50 mV.
Embodiment 36. The modified release formulation of any of Embodiments 19-35, wherein about 10-50% of the second active agent is released over a burst release period of about 24 hours to about 4 days.
Embodiment 37. The modified release formulation of any of Embodiments 19-36, wherein about 50-90% of the first active agent is released over a period of about 30 days.
Embodiment 38. The modified release formulation of any of Embodiments 19-37, in the form of a solution, gel, dispersion, or suspension.
Embodiment 39. The modified release formulation of any of Embodiments 19-37, which is a solid or liquid dosage form, such as dry powder, tablets, capsules, solution, gel, dispersion or suspension, etc.
Embodiment 40. A method of preparing the nanoparticles according to any of Embodiments 1-18, the method comprising mixing the cationic chitosan and the anionic cross-linker in an aqueous solution.
Embodiment 41. The method of Embodiment 40, wherein the mixing comprises stirring the cationic chitosan and the anionic cross-linker in the aqueous solution at a speed of about 100-1500 rpm for a period of about 15 minutes to 24 hours.
Embodiment 42. The method of Embodiment 40, wherein the mixing comprises mixing a solution of the cationic chitosan and a solution of the anionic cross-linker in a microfluid system.
Embodiment 43. The nanoparticles prepared by any of the methods according to Embodiments 40-42.
Embodiment 44. A method of preparing the modified release formulation according to any of Embodiments 19-39, the method comprising (1) mixing the cationic chitosan, the anionic cross-linker, and the first active agent to form core-shelled nanoparticles encapsulating the first active agent; (2) mixing the core-shelled nanoparticles obtained in (1) with the second active agent to form the drug-loaded nanoparticles with the second active agent adsorbed on the surface of the drug-loaded nanoparticles; and (3) coating the drug-loaded nanoparticles.
Embodiment 45. The modified release formulation obtained by the method according to Embodiment 44.
Embodiment 46. A method of stabilizing an active agent for storage comprising (1) mixing a cationic chitosan, an anionic cross-linker, and the active agent to form core-shelled nanoparticles encapsulating the active agent; and optionally (2) coating the core-shelled nanoparticles obtained in (1).
Embodiment 47. The method of Embodiment 46, wherein the active agent is a negatively charged agent, such as a negatively charged protein, antigen, drug molecules, antibodies, etc.
Embodiment 48. A method of delivering one or more active agents to a subject in need thereof, the method comprising administering to the subject the modified release formulation according to any of Embodiments 19-39 and 45.
Embodiment 49. The method of Embodiment 48, wherein the administering comprises intramuscular or subcutaneous injection of the modified release formulation.
Embodiment 50. The method of Embodiment 48, wherein the administration of the modified release formulation is through transdermal or transmucosal route, such as oral or intranasal.
Embodiment 51. A method of delivering a vaccine to a subject in need thereof, the method comprising administering the subject the pharmaceutical composition according to any of Embodiments, 1-18 or the modified release formulation according to any of Embodiments 19-35 and 45, wherein the first active agent and the second active agent are antigens derived from or corresponding to an infectious agent or a cancer.
Embodiment 52. A method of delivering therapeutic agents to a subject in need thereof, the method comprising administering the subject the pharmaceutical composition according to any of Embodiments, 1-18 or the modified release formulation according to any of Embodiments 19-35 and 45, wherein the first active agent and the second active agent are therapeutic agents.
Embodiment 53. The method of Embodiment 51 or 52, wherein the administering comprises intramuscular or subcutaneous injection of the pharmaceutical composition or modified release formulation.
Embodiment 54. The method of Embodiment 51 or 52, wherein the administration of the pharmaceutical composition or modified release formulation is through transdermal or transmucosal route, such as oral or intranasal.

It is to be understood that both the foregoing summary and the following detailed description are exemplary and explanatory only, and are not restrictive of the invention herein.

### BRIEF DESCRIPTION OF THE FIGURES

FIG. 1 shows scanning electron microscopy (“SEM”) images of layer-by-layer (“LbL”) nanoparticles (“NP”s) adjuvant and LbL encapsulated protein antigen NPs. The bottom image is the main chemical structure of LbL NPs (crosslinked N-trimethyl chitosan (“TMC”) with tripolyphophate (“TPP”)).

FIG. 2 shows characteristic ¹H-NMR spectrum of chitosan (a) and N-trimethyl chitosan (TMC, b) in D₂O and (c) shows the integral of quaternary amino peak and ¹H peaks for the calculation of degree of quaternization (“DQ”) for the TMC sample.

FIG. 3 shows FTIR spectra of Chitosan and TMC.

FIG. 4 shows scanning electron microscope images of LbL NP formations at different reaction time (15, 30 and 60 min). 60 min was found to be the optimized time for the core-shell structural nanoparticle formation.

FIG. 5 shows size of TMC-TPP single nanoparticle (left) and self-assembly nanoparticle (right) at different reaction time and purification conditions.

FIG. 6 shows appearance of crosslinked TMC nanoparticles after freeze-drying (left) and re-dispersed in PBS solution at 5 mg/ml concentration (right).

FIG. 7 shows nanoparticle microfluid synthesis schematic. A) Flow geometry and junction part; (B) synthesis flow chart.

FIG. 8 shows zeta potential of NPs with different TMC:PSS ratio. PSS refers to polystyrene sulfonate.

FIG. 9 shows Dye Alexa Fluor 488 (green, on the shell) and Texas-red labelled BSA LbL loading on TMC-TPP NPs (red circle, within the core) by two approaches without (a) and with (b) second protection layer coating (Blue circle, outer layer). (c) UV-Vis spectra of two dye mixture. (d) UV-Vis spectra of free Texas-red labelled and Alexa Fluor labelled BSA in the supernatant during the purification after loading into the chitosan NPs.

FIG. 10 shows release profile of NP formulation with and without PSS as the protective layer.

FIG. 11 shows zeta potential value of each composition of polymer TMC:TPP:PSS with and without BSA loading.

FIG. 12 shows SEM images of TMC-TPP-PSS-BSA1 NPs and average size of NP-BSA1.

FIG. 13 shows release profile of NP formulation with and without PSS as the protective layer.

FIG. 14 shows dye-labelled protein release profiles for different formulations of LbL crosslinked TMC nanoparticles by tuning of amount of outside of PSS or HA protective layer (insert is the schematic for two layers of loading of dye labelled proteins by coated with protective layers).

FIG. 15 shows Dynamic Light Scattering (“DLS”) measure TMC-FTIC-peptide-TPP nanoparticle size for two repeated reactions.

FIG. 16 shows ELISA antigenicity test for comparison of released AMA (erythrocytic stage) antigen (a) and CSP (pre-erythrocytic stage) antigen (b) from TMC nanoparticles with corresponding malaria antigens.

FIG. 17 shows Microscopy images for crosslinked TMC-TPP NP encapsulated EGFP and mCherry mRNA transfection in-vitro in APC.

FIG. 18 shows mean body weight and organ weight changes over time after IM injection of two doses of chitosan-nanoparticles at concentration of 0-25 mg/kg in Sprague-Dawley rats.

FIG. 19 shows CSP of P. falciparum-specific T cell responses (IFN-γ ELISPOT) induced by immunization with the LbL formulations herein and compared with two other adjuvants ISA 72 and 7dw8-5 using in the vaccine formulations for BALB/c mice study by 2-dose intramuscular injection. Blue, red, green referred as three formulations, 1^{st}, 2^{nd} and 3^{rd} bars from left to right in each data set, respectively, purple, the last bar in each data set, is the NP.

FIG. 20 shows ELISA titer of anti-CSP as an example which were induced by 2 dose or 3 dose immunizations of different formulations herein incorporated with and without adjuvant ISA 720 and 7DW8-5. Each group has four mice, and the data were averaged from these 4 mouse sera. For each data set, left shows data related to formulations with CSP alone, right shows date related to formulations with CSP, AMA, and MSP.

### DETAILED DESCRIPTION

The present disclosure generally relates to the pharmaceutical compositions comprising chitosan based nanoparticles, more particularly chitosan nanoparticles having a core-shell structure, which be configured to allow layer-by-layer delivery of active agent(s).

In some embodiments, the present disclosure relates to modified chitosan nanoparticles delivery/adjuvant platform, such as a trimethyl-chitosan nanoparticle delivery/adjuvant platform, for subunit protein/peptide or DNA/RNA vaccine delivery, or another adjuvant/antigen delivery. The nanoparticle structure allows layer by layer (LbL) delivery of the payload in a controlled fashion. As shown in the Example's section, a representative layer-by-layer delivery platform was successfully constructed using a crosslinked N-trimethyl chitosan (TMC), prepared from crosslinking TMC with tripolyphosphate (TPP), which can load in a controlled fashion with antigens such as proteins, peptides and/or nucleic acids to provide drug-loaded nanoparticles. The surface of the drug-loaded nanoparticles can then be coated, such as with a thin protective layer of hyaluronic acid sodium salt (HA) or polystyrene sulfonate (PSS). The constructed nanoparticles can deliver one or more adjuvant and/or active agent(s), such as antigens, vaccines, small molecular drugs, proteins, peptides, adjuvants, nucleic acids, etc., layer by layer (LbL) in a controlled fashion. Parameters including TMC methylation degree, reaction times, nanoparticle size, surface charge, and the ratio between each component of the formulations were found to have an effect on the efficiency of the modified chitosan nanoparticle formulations, see e.g., Table 1 of the Examples section.

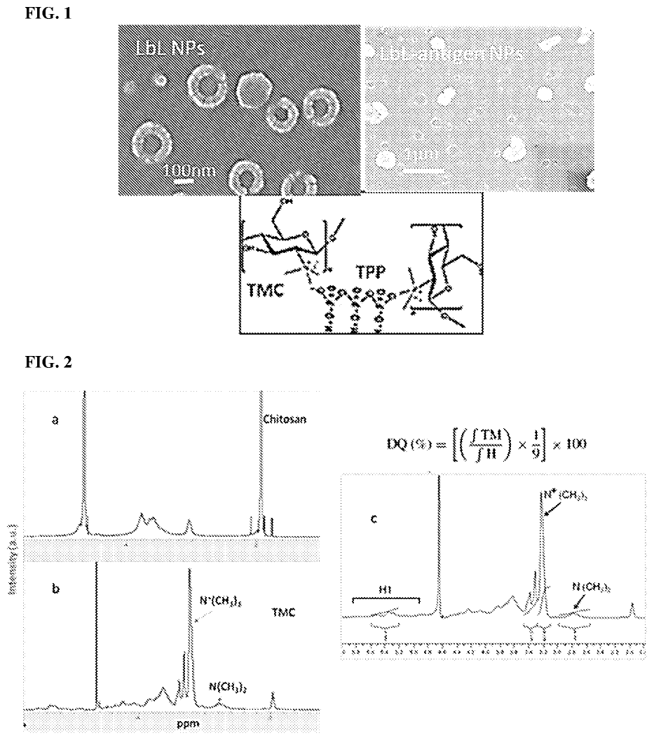

In one embodiment, in order to improve the solubility, biocompatibility, and interactions with antigen presenting cells, chitosan can be modified through trimethylation to generate surface charge variation and to form nanoparticles by ionic gelation (crosslinking) with tripolyphosphate (TPP) for antigen encapsulation (FIG. 1). The degree of trimethylation (surface charge) and particle size can be controlled for encapsulation and/or adsorption of different subunit antigen and/or nucleic acids. The adjuvant can self-assemble to encapsulate one or more antigens, with variable loading and dosing of antigens within one formulation. It can further stabilize the antigen (mRNA and protein) for long-term storage. Further, the delivery/adjuvant can be used for intramuscular and/or subcutaneous delivery and can also be extended to transdermal and mucosal deliveries (oral and/or intranasal). LbL NPs can be provided as a solution-based adjuvant or produced in other formats such as a dry powder or gel for different administration methods (liquid, tablets, capsules, sprays, gel or drops).

In some embodiments, the delivery technology (e.g., vaccine delivery technology) described herein is based on a modified chitosan biopolymer. The modified chitosan biopolymers herein typically are freely soluble in aqueous solution with a wide range of pH, exhibit sustained permeation through epithelial cells, and can improve accessibility of the antigens through penetrating cellular tight junctions, which results in significant benefits for mucosal/intranasal delivery. It allows easy entry into antigen-presenting cells (APCs), and thus significantly increases the utility of antigens and shortens the time to have effect (Lai 2014). This modified water-soluble chitosan biopolymer can be constructed to different mean nanoparticle sizes (90 nm to several μm) with crosslinker such as tripolyphosphate (TPP) in a core-shell type of structure, and the Zeta potential of NPs can be tuned, for example, from positive 50 mV to negative 40 mV to allow loading of multiple antigens at different presentation ratios. The chitosan NPs also have the potential to serve as an adjuvant by itself, acting synergistically to stimulate an immune response along with the high-density multiple antigen loading.

As shown in the Examples section, modified chitosan NPs have been demonstrated to provide an adjuvant effect in our development of a malaria vaccine. It also exhibited in vitro and in vivo stability and showed low cytotoxicity and systemic toxicity. Most importantly, the proof of concept was demonstrated that chitosan nanoparticles can serve as multiple antigen/protein encapsulation and the delivery vehicle with an ability to help induce both malaria Circumsporozoite protein (CSP) specific T-cell and humoral responses. Compared with two other commercially available adjuvants (Mondanide ISA 720 and 7DW8-5) for use as a malaria vaccine, intramuscular injection of LbL NP vaccine candidates induced the highest cellular response against Plasmodium falciparum Circumsporozoite protein (PfCSP).

In some embodiments, the present disclosure provides a pharmaceutical composition comprising modified chitosan nanoparticles. Typically, the modified chitosan nanoparticles comprise a crosslinked polymer containing a cationic chitosan and an anionic cross-linker. The modified chitosan nanoparticles herein typically have a core-shell structure, which is formed through self-assembly of the crosslinked polymer comprising the cationic chitosan and anionic cross-linker.

The modified chitosan nanoparticles typically have an average particle size of about 40 nm to about 1 μm, such as about 40 nm, about 50 nm, about 100 nm, about 150 nm, about 200 nm, about 300 nm, about 400 nm, about 500 nm, or about 1 μm, or any values or ranges between the recited values, such as about 40 nm to about 500 nm, about 100 nm to about 300 nm, about 150 nm to about 500 nm, about 200 nm to about 400 nm, etc., as determined by Dynamic Light Scattering. As used herein, unless otherwise specified as referring to single nanoparticles or unassembled nanoparticles, the average particle size of the modified chitosan nanoparticles should be understood as referring to the assembled nanoparticles having a core-shell structure, which may be optionally drug loaded. The single nanoparticles, i.e., in an unassembled state, typically have an average particle size of about 10-100 nm, such as about 15-80 nm, about 20-60 nm, about 50-70 nm, etc.

### Modified Chitosan Nanoparticles

It is important that the vector used for active agent delivery such as antigen delivery is highly stable or uniformly dispersed in the biological environment. The limited solubility of chitosan and chitosan-based materials therefore hinders its use and application for a wide range of biological environments.

The pharmaceutical compositions of the present disclosure typically include cationic chitosan, which has enhanced solubility in water across a wide range of pH as compared to unmodified chitosan. As understood in the art, chitosan is the product of complete or partial deacetylation of chitin and represents a polysaccharide of randomly distributed N-acetylglucosamine and glucosamine units. See e.g., Kritchenkov A. S. et al. Russ. Chem. Rev. 86:231 (2017), see also U.S. Pat. No. 7,740,883, WO96/20730, and U.S. Publication No. 2011/0158901. Typically, the proportion of glucosamine units in chitosan can range from 50% to 100%. As used herein, a cationic chitosan refers to a modified chitosan that contains quaternized ammonium cations and/or other cations, such that the modified chitosan is positively charged across a wide range of pH, such as 5-8. Thus, even though unmodified chitosan may become protonated, and thus positively charged, under certain acidic conditions, it is not the cationic chitosan as defined herein. In some preferred embodiments, the cationic chitosan can comprise quaternized ammonium cations, such as R—N(Me)₃+, wherein R is the residue of a chitosan. In some embodiments, the cationic chitosan can be water soluble (e.g., at least 10 mg/ml) at a neutral pH. In some preferred embodiments, the cationic chitosan can have an aqueous solubility of at least 10 mg/ml at a pH of 5-8. In some embodiments, the cationic chitosan can be water soluble (e.g., at least 10 mg/ml) in distilled water, in PBS solution, in alkaline or acidic aqueous solutions. In some preferred embodiments, the cationic chitosan herein is N-trimethylated chitosan (or TMC), i.e., the NH₂ group(s) of the glucosamine units in chitosan is trimethylated to form N(Me)₃⁺. Typically, the TMC herein can be soluble (e.g., at least 10 mg/ml) in distilled water, in PBS solution, and in alkaline or acidic aqueous solutions. It is believed that the solubility of TMC across the range of pH is due to the shifting in charge density originated by methylation of primary amino groups on chitosan.

In addition to improved solubility profiles, the positively charged cationic chitosan, such as TMC, can also be advantageous due to the high positive charge on the surface of crosslinked chitosan, such as crosslinked TMC herein. For example, these positive charges can be beneficial for loading of negatively charged active agents, such as proteins, peptides, or nucleic acids.

The degree of quaternization of the modified chitosan herein can be controlled to achieve a desired surface charge, solubility, and/or other desired properties. In some embodiments, the cationic chitosan, preferably TMC, is characterized by a degree of quaternization of between about 20% to about 60%, such as about 20-40%, about 20-50%, about 30-50%, about 40-60%, etc. The degree of quaternization can be determined by ¹H-NMR. The Examples section details a procedure for determining the degree of quaternization of TMC by using ¹H-NMR.

In some embodiments, the cationic chitosan is N-trimethylated chitosan (or TMC). TMC can be prepared by treating a chitosan with a methylating agent (e.g., MeI). Useful chitosan, prior to being methylated, is not particularly limited and include any of those known in the art, such as those commercially available. In some embodiments, the chitosan prior to being methylated can be characterized as (1) having a degree of deacetylation, such as 50% or above, for example, 60% or above, 70% or above, 80% or above, 90% or above, in particular, about 75-85%; and/or (2) an average viscosity molecular weight (Mᵥ) of about 50,000-190,000 Daltons, such as about 50,000-100,000, about 75,000-150,000, about 100,000-175,000 Daltons, etc.

The cationic chitosan is typically crosslinked with an anionic cross-linker in the pharmaceutical compositions herein. As used herein, an anionic cross-linker generally refers to a cross-linker that can become negatively charged at a pH of 7 or above. Typically, the anionic cross-linker includes one or more functional groups that can dissociate a proton, such as a —PO₃H group, so that at a pH of 7 or above, the one or more functional groups exist predominantly in anionic forms, such as —PO₃⁻. In some embodiments, the anionic cross-linker is tripolyphosphate.

In some preferred embodiments, the modified chitosan nanoparticles can comprise N-trimethylated chitosan (e.g., any of those described herein) and tripolyphosphate, with a weight ratio of N-trimethylated chitosan to tripolyphosphate ranging from about 2:1 to about 20:1, such as about 2:1, about 5:1, about 7:1, about 10:1, about 15:1, about 20:1, or any values or ranges between the recited values. Preferably, the weight ratio of N-trimethylated chitosan to tripolyphosphate ranges from about 5:1 to about 10:1, more preferably, about 5:1 to about 7:1.

The modified chitosan nanoparticles herein can optionally be coated with a coating layer. Typically, when coated, the coating layer comprises a negatively charged biocompatible polymer. As used herein, a negatively charged biocompatible polymer refers to a biocompatible polymer that is negatively charged at a pH of 7 and above. Typically, the negatively charged biocompatible polymer in its acid form contains one or more functional groups that can dissociate a proton, such as a —COOH or —SO₃H group etc., so that at a pH of 7 or above, the one or more functional groups exist predominantly in anionic forms, such as COO⁻ and/or SO₃⁻. Suitable negatively charged biocompatible polymer is not particular limited. Useful examples include hyaluronic acid salt (e.g., sodium hyaluronate) or polystyrene sulfonate (e.g., sodium polystyrene sulfonate). When coated, the coating layer is typically present in an amount such that the weight ratio of the cationic chitosan (e.g., N-trimethylated chitosan herein) to the coating layer is in the range of about 1:1 to about 200:1, for example, about 1:1, about 5:1, about 10:1, about 15:1, about 20:1, about 25:1, about 30:1, about 50:1, about 100:1, about 120:1, about 150:1, or any values or ranges between the recited values, such as about 5:1 to about 20:1, about 10:1 to about 25:1, about 5:1 to about 50:1, about 20:1 to about 100:1, about 15:1 to about 100:1, about 25:1 to about 120:1, etc. In some embodiments, the weight ratio of the cationic chitosan (e.g., N-trimethylated chitosan herein) to the coating layer can also be greater than 200:1, such as about 300:1 or greater, about 500:1 or greater, etc.

In some embodiments, the modified chitosan nanoparticles herein are coated with a coating layer, wherein (1) the nanoparticles comprise N-trimethylated chitosan and tripolyphosphate, with a weight ratio of N-trimethylated chitosan to tripolyphosphate ranging from about 2:1 to about 20:1, such as about 2:1, about 5:1, about 7:1, about 10:1, about 15:1, about 20:1, or any values or ranges between the recited values, preferably, about 5:1 to about 10:1, more preferably, about 5:1 to about 7:1; and (2) the coating layer comprises polystyrene sulfonate (e.g., sodium polystyrene sulfonate). In some embodiments, the weight ratio of N-trimethylated chitosan to polystyrene sulfonate ranges from about 1:1 to about 200:1, for example, about 1:1, about 5:1, about 10:1, about 15:1, about 20:1, about 25:1, about 30:1, about 50:1, about 100:1, about 120:1, about 150:1, or any values or ranges between the recited values, preferably about 5:1 to about 50:1, such as about 10:1 or 20:1. In some embodiments, the weight ratio of N-trimethylated chitosan to polystyrene sulfonate can also be greater than 200:1, such as about 300:1 or greater, about 500:1 or greater, etc. Useful polystyrene sulfonate for the pharmaceutical compositions herein is not particular limited and include any of those known in the art, such as those commercially available. For example, in some embodiments, suitable polystyrene sulfonate can be a poly(4-styrenesulfonic acid) sodium salt with an average M_{w} of about 70 k, commercially available from Sigma-Aldrich. Other grades of polystyrene sulfonate, including other salts (e.g., potassium salt, calcium salt, etc.) or at a different molecular weight, such as an average M_{w} of about 200 k or 1000 k, are also available and can be used for the pharmaceutical compositions herein. In some embodiments, the polystyrene sulfonate is sodium polystyrene sulfonate, with an average M_{w} of about 50 k to about 100 k.

In some embodiments, the modified chitosan nanoparticles herein are coated with a coating layer, wherein (1) the nanoparticles comprise N-trimethylated chitosan and tripolyphosphate, with a weight ratio of N-trimethylated chitosan to tripolyphosphate ranging from about 2:1 to about 20:1, such as about 2:1, about 5:1, about 7:1, about 10:1, about 15:1, about 20:1, or any values or ranges between the recited values, preferably, about 5:1 to about 10:1, more preferably, about 5:1 to about 7:1; and (2) the coating layer comprises hyaluronic acid salt (e.g., sodium hyaluronate). In some embodiments, the weight ratio of N-trimethylated chitosan to hyaluronic acid salt ranges from about 1:1 to about 200:1, for example, about 1:1, about 5:1, about 10:1, about 15:1, about 20:1, about 25:1, about 30:1, about 50:1, about 100:1, about 120:1, about 150:1, or any values or ranges between the recited values, preferably about 5:1 to about 50:1, such as about 10:1 or 20:1. In some embodiments, the weight ratio of N-trimethylated chitosan to hyaluronic acid salt can also be greater than 200:1, such as about 300:1 or greater, about 500:1 or greater, etc. Useful hyaluronic acid salt for the pharmaceutical compositions herein is also not particular limited and include any of those known in the art, such as those commercially available. For example, in some embodiments, the hyaluronic acid salt is sodium hyaluronate, with an average M_{w} of about 30 k to about 1000 k, such as about 60 k, about 100 k, about 600 k, about 1000 k, or any values or ranges between the recited values.

The modified chitosan nanoparticles can typically include at least one active agent, which can be encapsulated within the nanoparticles and/or adsorbed on the surface of the nanoparticles. For example, in some embodiments, at least a portion of the active agent can be in the core of the core-shell nanoparticles. In some embodiments, at least a portion of the active agent can be adsorbed on the surface of the nanoparticles.

As exemplified herein, the modified chitosan nanoparticles herein may also be used as an adjuvant. In some embodiments, the modified chitosan nanoparticles herein can also include no active agent and be included in a formulation as an adjuvant.

Suitable active agents (including prodrugs and may alternatively be referred to herein as drugs) for the pharmaceutical composition herein are not particularly limited and include both therapeutic agents and prophylactic agents, such as an antigen or vaccine. To be clear, active agents as used herein can also include an adjuvant, other than the adjuvant that the modified chitosan nanoparticles are functioning as. For example, in some embodiments, the active agent can be a small molecule drug, a biologic drug, an antigen (e.g., for a vaccine), a nucleic acid such as an oligonucleotide, polynucleotide, DNA, RNA, a silencing RNA (e.g., small interfering RNA (siRNA), microRNA (miRNA), and short hairpin RNA (shRNA)), antisense RNA and ribozymes), mRNA, a protein, a polypeptide such as an antibody, an antigen binding fragment of an antibody, a single domain antibody (VHH), an aptamer, a protein having an alternative binding scaffold, a peptide, a glycosaminoglycan, an oligosaccharide, or a polysaccharide, or a derivative or analog thereof. In some embodiments, the active agent can be an adjuvant, such as glycolipid adjuvant 4-Fluorophenylundecanoyl-alpha-galactosylceramide (7DW8-5) or other synthetic analog of α-galactosylceramide (α-GalCer). In some embodiments, the active agent is not an adjuvant. Preferably, the active agent is negatively charged (PI<7) at pH 7 or higher. In some preferred embodiments, the active agent can be a hydrophobic molecule, which can be absorbed by hydrophobic interaction, such as a small molecule drug having a Log P of greater than 1, e.g., 1-5. A small molecule drug generally refers to a drug that has a molecular weight of less than 1,000 Daltons, preferably, less than 500 Daltons. In some embodiments, the nucleic acid can be a RNA, such as an mRNA. In some embodiments, the nucleic acid can be a DNA.

In some embodiments, the modified chitosan nanoparticles can also include other pharmaceutically acceptable excipients. For example, in some embodiments, the modified chitosan nanoparticles can include a surfactant, such as a non-ionic surfactant, e.g., poloxamers such as poloxamer 124, poloxamer 188, poloxamer 237, poloxamer 338, poloxamer 407; and poly(oxyethyl)-sorbitan monooleates (“polysorbates”) such as polysorbate 20 (Tween™ 20), polysorbate 60 (Tween™ 60), polysorbate 80 (Tween™ 80); or any combination thereof. In further embodiments, the modified chitosan nanoparticles include polysorbate 80 (Tween™ 80).

The modified chitosan nanoparticles herein can typically have a zeta potential ranging from about −40 mV to about 50 mV. The surface charge of the modified chitosan nanoparticles can depend on various factors, for example, the amount of anionic cross-linker, the type and amount of drug loaded, and whether a coating layer is present and the amount thereof. Typically, the cationic chitosan nanoparticles prior to cross linking are highly positively charged, with a zeta potential greater than 30 mV, such as about 30-60 mV, about 40-50 mV, such as bout 45 mV, about 35-55 mV, etc. Upon cross linking with an anionic cross-linker herein, such as with TPP, the nanoparticles typically have a zeta potential ranges from about 5-50 mV, such as about 5-35 mV, about 10-40 mV, about 15-25 mV, about 15-35 mV, etc. The zeta potential of the modified chitosan nanoparticles can be even further lowered when a negatively charged drug is loaded on the surface and/or the nanoparticles are coated with a negatively charged biopolymer herein.

### Layer-by-Layer Delivery

The provided formulations may be of any order release kinetics, including zero-order release, first-order release, second-order release, delayed release, sustained release, immediate release, and any combination thereof. Some embodiments of the present disclosure are directed to a modified release formulation which can optionally be configured to allow layer-by-layer delivery of one or more active agents in a controlled fashion. The term “modified release” is used herein to distinguish an immediately release profile and unless contradictory from context, generally encompasses those release profiles which are not immediate release. “The term “sustained release” (also referred to as “extended release”) is used in its conventional sense to refer to a drug formulation that provides for gradual release of a drug over an extended period of time, and that preferably, although not necessarily, results in substantially constant blood levels of a drug over an extended time period. The term “delayed release” is used in its conventional sense to refer to a drug formulation in which there is a time delay between administration of the formulation and the release of the drug there from. “Delayed release” may or may not involve gradual release of drug over an extended period of time, and thus may or may not be “sustained release.” Thus, “modified release” can encompass for example, delayed release and/or sustained release unless context dictates otherwise. In some embodiments, modified release formulation refers to a delayed (i.e., non-immediate) release formulation. In some embodiments, a modified release formulation refers to a sustained release formulation.

In some embodiments, the modified release formulation can comprise:
(1) drug-loaded nanoparticles having a core-shell structure, wherein the drug-loaded nanoparticles comprise a crosslinked polymer comprising a cationic chitosan and an anionic cross-linker, a first active agent, and a second active agent; and
(2) a layer coating the drug-loaded nanoparticles.
   The first active agent is typically encapsulated within the drug-loaded nanoparticles, such as present in the core section of the core-shell structured nanoparticles. On the other hand, the second active agent is typically adsorbed on the surface of the drug-loaded nanoparticles. The first and second active agents can be the same or different active agents.

Typically, the drug-loaded nanoparticles have an average particle size of about 40 nm to about 1 μm, such as about 40 nm, about 50 nm, about 100 nm, about 150 nm, about 200 nm, about 300 nm, about 400 nm, about 500 nm, or about 1 μm, or any values or ranges between the recited values, such as about 40 nm to about 500 nm, about 100 nm to about 300 nm, about 150 nm to about 500 nm, about 200 nm to about 400 nm, etc., as determined by Dynamic Light Scattering. In some preferred embodiments, the drug-loaded nanoparticles have an average particle size of about 100 nm to about 500 nm, such as about 200 nm to about 400 nm. As used herein, unless otherwise specified or obviously contrary from context, the average particle size of the drug-loaded nanoparticles refers to that of the drug-loaded nanoparticles without considering the coating layer.

The crosslinked polymer, cationic chitosan, and anionic cross-linker suitable for use in the drug-loaded nanoparticles include any of those described herein. For example, in some preferred embodiments, the drug-loaded nanoparticles can comprise a crosslinked polymer of N-trimethylated chitosan (e.g., any of those described herein) and tripolyphosphate, wherein a weight ratio of N-trimethylated chitosan to tripolyphosphate ranges from about 2:1 to about 20:1, such as about 2:1, about 5:1, about 7:1, about 10:1, about 15:1, about 20:1, or any values or ranges between the recited values. Preferably, the weight ratio of N-trimethylated chitosan to tripolyphosphate ranges from about 5:1 to about 10:1, more preferably, about 5:1 to about 7:1.

The layer coating the drug-loaded nanoparticles is also not particularly limited and includes any of the coating layers described herein. For example, in some embodiments, the drug-loaded nanoparticles comprise N-trimethylated chitosan and tripolyphosphate, with a weight ratio of N-trimethylated chitosan to tripolyphosphate ranging from about 2:1 to about 20:1, such as about 2:1, about 5:1, about 7:1, about 10:1, about 15:1, about 20:1, or any values or ranges between the recited values, preferably, about 5:1 to about 10:1, more preferably, about 5:1 to about 7:1; and the coating layer comprises polystyrene sulfonate (e.g., sodium polystyrene sulfonate). In some embodiments, the weight ratio of N-trimethylated chitosan to polystyrene sulfonate ranges from about 1:1 to about 200:1, for example, about 1:1, about 5:1, about 10:1, about 15:1, about 20:1, about 25:1, about 30:1, about 50:1, about 100:1, about 120:1, about 150:1, or any values or ranges between the recited values, preferably about 5:1 to about 50:1, such as about 10:1 or 20:1. In some embodiments, the drug-loaded nanoparticles comprise N-trimethylated chitosan and tripolyphosphate, with a weight ratio of N-trimethylated chitosan to tripolyphosphate ranging from about 2:1 to about 20:1, such as about 2:1, about 5:1, about 7:1, about 10:1, about 15:1, about 20:1, or any values or ranges between the recited values, preferably, about 5:1 to about 10:1, more preferably, about 5:1 to about 7:1; and the coating layer comprises hyaluronic acid salt (e.g., sodium hyaluronate). In some embodiments, the weight ratio of N-trimethylated chitosan to hyaluronic acid salt ranges from about 1:1 to about 200:1, for example, about 1:1, about 5:1, about 10:1, about 15:1, about 20:1, about 25:1, about 30:1, about 50:1, about 100:1, about 120:1, about 150:1, or any values or ranges between the recited values, preferably about 5:1 to about 50:1, such as about 10:1 or 20:1.

In some embodiments, the drug-loaded nanoparticles and/or coating layer can also include other pharmaceutically acceptable excipients. For example, in some embodiments, the drug-loaded nanoparticles can include a surfactant, such as a non-ionic surfactant, e.g., poloxamers such as poloxamer 124, poloxamer 188, poloxamer 237, poloxamer 338, poloxamer 407; and poly(oxyethyl)-sorbitan monooleates (“polysorbates”) such as polysorbate 20 (Tween™ 20), polysorbate 60 (Tween™ 60), polysorbate 80 (Tween™ 80); or any combination thereof. In further embodiments, the modified chitosan nanoparticles include polysorbate 80 (Tween™ 80).

The coated drug-loaded nanoparticles herein can typically have a zeta potential ranging from about −40 mV to about 50 mV. In some preferred embodiments, the coated drug-loaded nanoparticles can have a positive zeta potential, which is believed to be beneficial for the nanoparticles to reach certain desired targets/cells. In some embodiments, the coated drug-loaded nanoparticles can have a zeta potential ranging from about 5-50 mV, such as about 5-35 mV, about 10-40 mV, about 15-25 mV, about 15-35 mV, etc. The zeta potential of the coated drug-loaded nanoparticles herein can be tuned, for example, by modifying the amount and type of the coating.

Suitable first and second active agents for the modified release formulations herein are also not particularly limited. For example, in some embodiments, the first and second active agents can be independently a small molecule drug, a biologic drug, an antigen (e.g., for a vaccine), a nucleic acid such as an oligonucleotide, polynucleotide, DNA, RNA, a silencing RNA (e.g., small interfering RNA (siRNA), microRNA (miRNA), and short hairpin RNA (shRNA)), antisense RNA and ribozymes), mRNA, a protein, a polypeptide such as an antibody, an antigen binding fragment of an antibody, a single domain antibody (VHH), an aptamer, a protein having an alternative binding scaffold, a peptide, a glycosaminoglycan, an oligosaccharide, or a polysaccharide, or a derivative or analog thereof, or an adjuvant such as glycolipid adjuvant 4-Fluorophcnylundecanoyl-alpha-galactosylceramide (7DW8-5) or other synthetic analog of α-galactosylceramide (α-GalCer). In some embodiments, the first and/or second active agent is a therapeutic agent. In some embodiments, the first and/or second active agent is an agent useful in treating or preventing cancer. In some embodiments, the first and/or second active agent is a chemotherapeutic agent. In some embodiments, the first and/or second active agent is an agent useful in treating or preventing an infectious disease (e.g., an anti-microbial, anti-viral, anti-fungal, anti-parasitic, anti-protozoan, or anti-helminth agent). In some embodiments, the first and/or second active agent is an immunomodulatory (e.g., an immunostimulant or immunosuppressant). Preferably, the first and/or second active agents is negatively charged (PI<7) at pH 7 or higher, e.g., both the first and second active agents are negatively charged (PI<7) at pH 7 or higher. In some preferred embodiments, the first and/or second active agents can be a hydrophobic molecule, which can be absorbed by hydrophobic interaction, such as a small molecule drug having a Log P of greater than 1, e.g., 1-5. In some embodiments, both the first active agent and the second active agent are proteins, which may be the same or different. In some embodiments, the first and second active agents are both antigens, which may be the same or different. In some embodiments, the first and second active agents are the same. In some embodiments, the first and second active agents are different, preferably, both are useful for treating or preventing the same disease, disorder, or condition. For example, in some embodiments, the first and second active agents are different, and it is beneficial to deliver the first and second active agents at a different rate, for example, a slower delivery of the first active agent is deemed beneficial. In some embodiments, it is beneficial to prolong the release of the first and second active agents, same or different, to achieve an extended therapeutic or prophylactic effect. In some embodiments, one of the first and second active agents is an adjuvant such as glycolipid adjuvant 4-Fluorophenylundecanoyl-alpha-galactosylceramide (7DW8-5) or other synthetic analog of α-galactosylceramide (α-GalCer), and the other of the first and second active agents is as defined herein, such as an antigen, vaccine, etc.

The amount of the first and second active agents can vary, typically in a total amount of about 10-100% (e.g., about 20%, about 30%, about 40%, about 50%, about 60%, about 70%, about 80%, about 90%, about 100%, or any range or value between the recited values) by weight of the cationic chitosan.

As exemplified herein, by adjusting the coating layer, the release rate of the first and/or second active agents can be adjusted. In some embodiments, the coating layer can be adjusted so that about 10-50% of the second active agent is released over a burst release period of about 24 hours to about 4 days. In some embodiments, the coating layer is adjusted such that about 50-90% of the first active agent is released over a period of about 30 days. A higher amount of coating layer (such as polystyrene sulfonate coating or sodium hyaluronate coating at a weight ratio to the TMC of about 1:20) tends to prolong the release of the first active agent. In some embodiments, the coating layer can be adjusted so that less than 50%. such as less than 40%, less than 20%, etc., of the first active agent is released over a period of about 30 days.

In some embodiments, a third active agent can be included in the modified release formulation, such as adsorbed to the coating layer. The third active agent can be the same or different from the first and/or second active agent. Similarly, the third active agent can be a small molecule drug, a biologic drug, an antigen (e.g., for a vaccine), a nucleic acid such as an oligonucleotide, polynucleotide, DNA, RNA, a silencing RNA (e.g., small interfering RNA (siRNA), microRNA (miRNA), and short hairpin RNA (shRNA)), antisense RNA and a ribozyme), mRNA, a protein, a polypeptide such as an antibody, an antigen binding fragment of an antibody, a single domain antibody (VHH), an aptamer, a proteins having an alternative binding scaffold, a peptides, an glycosaminoglycans, an oligosaccharide, or a polysaccharide, or a derivative or analog thereof. In some embodiments, the third active agent can be an adjuvant such as glycolipid adjuvant 4-Fluorophenylundecanoyl-alpha-galactosylceramide (7DW8-5) or other synthetic analog of α-galactosylceramide (α-GalCer). In sum embodiments, the third active agent is a therapeutic agent. In some embodiment, the third active agent is included in the formulation without being particularly associated with the coated drug-loaded nanoparticles.

In some embodiments, more than one coating layers can be applied to the drug-loaded nanoparticles. For example, in some embodiments, a third active agent is adsorbed on the inner coating layer, and then a second coating layer can be applied to encapsulate the third active agent.

The modified release formulation herein can additionally include pharmaceutically acceptable excipients, carriers, etc., which are not particularly limited. For example, in some embodiments, the modified release formulation herein can be formulated in the form of a solution, gel, dispersion, or suspension. In some embodiments, the modified release formulation herein can be formulated in the form of solid or liquid dosage form, such as dry powder, tablets, capsules, solution, gel, dispersion or suspension, etc. Excipients useful for formulating solid or liquid dosage form are generally known in the art. Non-limiting suitable excipients include, for example, encapsulating materials or additives such as absorption accelerators, antioxidants, binders, buffers, carriers, coating agents, coloring agents, diluents, disintegrating agents, emulsifiers, extenders, fillers, flavoring agents, humectants, lubricants, perfumes, preservatives, propellants, releasing agents, sterilizing agents, sweeteners, solubilizers, wetting agents and mixtures thereof. See also Remington's The Science and Practice of Pharmacy, 21st Edition, A. R. Gennaro (Lippincott, Williams & Wilkins, Baltimore, Md., 2005; incorporated herein by reference), which discloses various excipients used in formulating pharmaceutical compositions and known techniques for the preparation thereof.

The modified release formulation can be used for delivering of one or more active agents (e.g., the first, second, and/or third active agents, etc.) to a subject in need thereof, such as those in need of treatment or prevention of a disease or disorder (e.g., malaria, infections caused by tuberculosis, infections caused by HIV, influenza, or a coronavirus (e.g., severe acute respiratory syndrome coronavirus 2 (SARS-CoV-2), coronavirus disease 2019 (COVID-19), etc.). Typically, the method comprises administering to the subject an effective amount of the modified release formulation. The route of administration is not particularly limited and can include any of those known in the art. For example, in some embodiments, the administering comprises intramuscular or subcutaneous injection of the modified release formulation. In some embodiments, the administration of the modified release formulation is through transdermal or transmucosal route, such as oral or intranasal. The effective amount of the active agent(s) can depend on the recipient of the treatment, the disease or disorder being treated, or targeted for prevention, and the severity thereof, the composition containing the active agent(s), the time of administration, the route of administration, the duration of treatment, potency of the active agent(s) (e.g., for inducing immune responses), its rate of clearance and whether or not another drug is co-administered.

### Method of Preparation

Some embodiments of the present disclosure are directed to methods of preparing the pharmaceutical compositions or modified release formulations herein.

In some embodiments, the present disclosure provides a method of preparing nanoparticles having a core-shell structure herein. Typically, the method comprises mixing a cationic chitosan (e.g., N-trimethylated chitosan herein) and an anionic cross-linker (e.g., described herein, such as TPP) in an aqueous solution.

Suitable methods of mixing are not particularly limited. In some embodiments, the mixing can comprise stirring the cationic chitosan (e.g., N-trimethylated chitosan herein) and the anionic cross-linker (e.g., described herein, such as TPP) in the aqueous solution, for example, at a speed of about 100-1500 rpm (e.g., about 200 rpm, 400 rpm, 700 rpm, 1200 rpm, 1500 rpm, or any values and ranges between the recited values) for a period of time, such as about 15 minutes to 24 hours (e.g., about 1 hour, about 4 hours, about 8 hours, about 12 hours, or about 24 hours, or any values and ranges between the recited values). In some embodiments, the mixing can also comprise mixing a solution of the cationic chitosan (e.g., N-trimethylated chitosan herein) and a solution of the anionic cross-linker (e.g., described herein, such as TPP) in a microfluid system. Exemplified procedures using the stirring method and microfluid system are shown in the Examples section.

The ratios of the cationic chitosan and anionic cross-linker include any of those described herein, such as those shown in Table 1 of the Examples section. Typically, under the reaction conditions, the nanoparticles can self-assemble to provide nanoparticles having a core-shell structure.

In some embodiments, the present disclosure provides a method of preparing a modified release formulation containing drug-loaded nanoparticles herein. In some embodiments, the method comprises (1) mixing a cationic chitosan, an anionic cross-linker, and a first active agent to form core-shelled nanoparticles encapsulating the first active agent; (2) mixing the core-shelled nanoparticles obtained in (1) with a second active agent to form the drug-loaded nanoparticles with the second active agent adsorbed on the surface of the drug-loaded nanoparticles; and (3) coating the drug-loaded nanoparticles. Suitable methods of mixing are not particularly limited. In some embodiments, the mixing in (1) can comprise stirring the cationic chitosan (e.g., N-trimethylated chitosan herein), the anionic cross-linker (e.g., described herein, such as TPP), and the first active agent in the aqueous solution, for example, at a speed of about 100-1500 rpm (e.g., about 200 rpm, 400 rpm, 700 rpm, 1200 rpm, 1500 rpm, or any values and ranges between the recited values) for a period of time, such as about 15 minutes to 24 hours (e.g., about 1 hour, about 4 hours, about 8 hours, about 12 hours, or about 24 hours, or any values and ranges between the recited values). In some embodiments, the mixing in (1) can also comprise mixing a solution of the cationic chitosan (e.g., N-trimethylated chitosan herein), a solution of the anionic cross-linker (e.g., described herein, such as TPP), and a solution of the first active agent in a microfluid system. Exemplified procedures using the stirring method and microfluid system are shown in the Examples section. Various coatings are also suitable. For example, in some embodiments, the coating in (3) comprises coating the drug-loaded nanoparticles with a negatively charged biocompatible polymer herein. The cationic chitosan, anionic cross-linker, coating, and amounts/ratios thereof include any of those described herein, such as those shown in Table 1 of the Examples section. The first and second active agents, as well as amount thereof, can also include any of those described herein.

In some embodiments, the present disclosure also provides a method of stabilizing an active agent for storage comprising (1) mixing a cationic chitosan, an anionic cross-linker, and the active agent to form core-shelled nanoparticles encapsulating the active agent; and optionally (2) coating the core-shelled nanoparticles obtained in (1). Suitable methods of mixing are not particularly limited. In some embodiments, the mixing in (1) can comprise stirring the cationic chitosan (e.g., N-trimethylated chitosan herein), the anionic cross-linker (e.g., described herein, such as TPP), and the active agent in an aqueous solution, for example, at a speed of about 100-1500 rpm (e.g., about 200 rpm, 400 rpm, 700 rpm, 1200 rpm, 1500 rpm, or any values and ranges between the recited values) for a period of time, such as about 15 minutes to 24 hours (e.g., about 1 hour, about 4 hours, about 8 hours, about 12 hours, or about 24 hours, or any values and ranges between the recited values). In some embodiments, the mixing in (1) can also comprise mixing a solution of the cationic chitosan (e.g., N-trimethylated chitosan herein), a solution of the anionic cross-linker (e.g., described herein, such as TPP), and a solution of the active agent in a microfluid system. Exemplified procedures using the stirring method and microfluid system are shown in the Examples section. Various coatings are also suitable. For example, in some embodiments, the coating comprises coating the drug-loaded nanoparticles with a negatively charged biocompatible polymer herein. The cationic chitosan, anionic cross-linker, coating, and amounts/ratios thereof include any of those described herein, such as those shown in Table 1 of the Examples section. The active agent is typically, a negatively charged agent (PI<7), such as a negatively charged protein, antigen, drug molecules, antibodies, etc. In some embodiments, the active agent can also include a hydrophobic molecule, such as a small molecule drug having a Log P of at least 1, e.g., 1-5. Typically, stabilities of the active agent in a formulation prepared according to the methods herein are better than an otherwise similar formulation except without the cross-linked cationic chitosan nanoparticles and the optional coating.

### Drug-Loaded Nanoparticles

In some embodiments, the disclosure provides a composition comprising a drug-loaded nanoparticle that comprises one or more active agents. In some embodiments, the drug-loaded nanoparticle comprises 1, 2, 3, 4, 5, or more than 5 different active agents. In some embodiments, the drug-loaded nanoparticle comprises 1-5, 1-10, 1-15, 1-20, 1-25, 1-30, 2-10, 2-15, 2-20, 2-25, 3-10, 3-15, 3-20, 3-25, 4-10, 4-15, 4-20, 4-25, 5-10, 5-15, 5-20, or 5-25 different active agents. In some embodiments, the drug-loaded nanoparticle comprises at least one active agent that is a therapeutic agent.

The terms “drug” and “active agent” are used interchangeably herein. Drug(s)/active agent(s) include any one or more of biologically active agents, therapeutic agents, and/or diagnostic agents unless context dictates otherwise. Exemplary active agents that may be contained in a provided drug-loaded nanoparticle include without limitation, small molecules, biologics, antigens, nucleic acids such as oligonucleotides, polynucleotides, DNA, RNA, a silencing RNA (e.g., small interfering RNA (siRNA) and microRNA (miRNA), and short hairpin RNA (shRNA), antisense RNA and ribozymes), mRNA, proteins, polypeptides such as antibodies, antigen binding fragments of antibodies, single domain antibodies (VHH), aptamers, proteins having alternative binding scaffolds, peptides, glycosaminoglycans, oligosaccharides, and polysaccharides, and derivatives or analogs thereof. In some embodiments, active agents can also be an adjuvant. In some embodiments, active agents do not include an adjuvant.

In some embodiments, the disclosure provides a vaccine composition containing a drug-loaded nanoparticle that comprises one or more different antigens. In some embodiments, the drug-loaded nanoparticle comprises 1, 2, 3, 4, 5, or more than 5 different antigens. In some embodiments, the drug-loaded nanoparticle comprises 1-5, 1-10, 1-15, 1-20, 1-25, 1-30, 2-10, 2-15, 2-20, 2-25, 3-10, 3-15, 3-20, 3-25, 4-10, 4-15, 4-20, 4-25, 5-10, 5-15, 5-20, or 5-25 different antigens. As used herein, the term “antigen” refers to a substance that can induce an immune response a subject. Suitable antigens of the composition are those that are capable of inducing a humoral immune response in a subject. “Antigen” also includes a polynucleotide that encodes the polypeptide that functions as an antigen. Nucleic acid-based vaccination strategies are known, wherein a vaccine composition that contains a polynucleotide is administered to a subject. The antigenic polypeptide encoded by the polynucleotide is expressed in the subject, such that the antigenic polypeptide is ultimately present in the subject, just as if the vaccine composition itself had contained the polypeptide.

In some embodiments, the disclosure provides a vaccine composition containing a drug-loaded nanoparticle that comprises one or more antigens derived from or corresponding to an infectious agent. In some embodiments, the drug-loaded nanoparticle comprises 1, 2, 3, 4, 5, or more than 5 different antigens derived from or corresponding to an infectious agent. In some embodiments, the drug-loaded nanoparticle comprises 1-5, 1-10, 1-15, 1-20, 1-25. 1-30, 2-10, 2-15, 2-20, 2-25, 3-10, 3-15, 3-20, 3-25, 4-10, 4-15, 4-20, 4-25, 5-10, 5-15, 5-20, or 5-25 different antigens derived from or corresponding to an infectious agent. In further embodiments, the one or more antigens are derived from of correspond to an antigen from an infectious agent that is a virus, a bacteria, a fungus, a protozoan, a parasite, and/or a helminth. Polypeptides or fragments thereof that may be useful as antigens in the provided drug-loaded nanoparticles include, without limitation, those derived from or corresponding to cholera toxoid, tetanus toxoid, diphtheria toxoid, pertussis toxoid, hepatitis B surface antigen, hemagglutinin (e.g. H5N1 recombinant hemagglutinin protein), anthrax recombinant protective antigen, neuraminidase, influenza M protein, CSP, PfSSP2, LSA-1, MSA-1, SERA, AMA-1, Pfs25, Pfg27, PfHRP2, PfHRP3, pLDH, MSP1, MSP2, Der-p-1, and/or Der-f-1.

In some embodiments, the disclosure provides a vaccine composition containing a drug-loaded nanoparticle that comprises antigens that are derived from or correspond to antigens expressed during different lifecycle stages of an infectious agent. For example, in some embodiments, the drug-loaded nanoparticles contain antigens derived from or corresponding to antigens expressed during two or more of the sporozoite stage, blood stage, liver stage, or sexual stage of the malaria plasmodium parasite. In further embodiments the drug-loaded nanoparticles contain 1, 2, 3, 4 or more antigens corresponding to the CSP and PfSSP2 sporozoite proteins, the LSA-1 liver stage protein, the MSA-1, MSP-1, SERA, and AMA-1 blood stage proteins, and the Pfs25 sexual stage protein of plasmodium. In some embodiments, the provided drug-loaded nanoparticles comprises antigens that are derived from or correspond to plasmodium CSP, AMA1, and MSP1.

In some embodiments, the disclosure provides a vaccine composition containing a drug-loaded nanoparticle that comprises two or more antigens derived from or corresponding to antigens expressed during two or more phases of an infectious disease. For example, in some embodiments, the drug-loaded nanoparticles contain antigens derived from or corresponding to antigens expressed by M. tuberculosis during 2 or more of the infectious phase, latent phase, and reactivation phase of M. tuberculosis infection

In some embodiments, the provided drug-loaded nanoparticle comprises one or more antigens (e.g., 1, 2, 3, 4, 5, or more than 5 different antigens, or 1-5, 1-10, 1-15, 1-20, 1-25, 1-30, 2-10, 2-15, 2-20, 2-25, 3-10, 3-15, 3-20, 3-25, 4-10, 4-15, 4-20, 4-25, 5-10, 5-15, 5-20, or 5-25 different antigens) that is derived from or corresponds to an antigen(s) expressed by a cancer. In some embodiments, one or more antigens is a Tumor-Associated Antigen (TAA). As used herein, a TAA is an antigen derived from or corresponding to an aberrantly overexpressed self-antigens in a tumor cell compared to a normal cell and might represent a universal antigen among patients with the same malignancy. TAAs can also include: cell lineage differentiation antigens, which are normally not expressed in adult tissue (e.g., tyrosinase, gp100, MART-1, prostate-specific antigen (PSA); prostatic acid phosphatase (PAP)); and cancer/germline antigens (also known as cancer/testis), which are normally expressed only in immune privileged germline cells (e.g., MAGE-A1, MAGE-A3, NY-ESO-1, and PRAME). In some embodiments, one or more antigens is a Tumor-Specific Antigen (TSA). As used herein, a TSA is an antigen that is specific to tumors and is not expressed on the surface of normal cells. A TSA can include for example, a mutated neoantigen as well as an antigen from an oncovirus, and an endogenous retroviral element (HERV). In some embodiments, the drug-loaded nanoparticle comprises one or more antigens that is a TAA and one or more antigens that is a TSA.

Polypeptides or fragments thereof that may be useful as antigens in the provided drug-loaded nanoparticles include, without limitation, those derived from or corresponding to a TAA or TSA expressed in colorectal cancer, gastric cancers, urothelial/bladder cancer, pancreatic cancer, breast cancer (e.g., TNBC), ovarian cancer, prostate cancer, liver cancer (e.g., HCC), kidney, lung cancer (e.g., NSCLC and SCLC), melanoma, glioblastoma, myeloma (e.g., SPCM), leukemia (lympocytic leukemia), or lypmphoma (ALL, follicular lymphoma.

Additional polypeptides or fragments thereof that may be useful as antigens in the provided drug-loaded nanoparticles include, without limitation, those derived from or corresponding to aldolase, adipophilin, AFP, AIM-2, ART-4, BAGE, α-fetoprotein, BCL-2, Bcr-Abl, BING-4, CEA, CPSF, CT, cyclin D1, Ep-CAM, EphA2, EphA3, ELF-2, FGF-5, G250, Gonadotropin Releasing Hormone, gp100, HER-2, intestinal carboxyl esterase (iCE), HIF-1α, IGF-1R, IGFBP-2, IL13Rα2, MAGE-1, MAGE-2, MAGE-3, MAGE-A1, MAGE-A3, MART-1, MART-2, M-CSF, MDM-2, mesothelin, MMP-2, MUC-1, NY-ESO-1, MUM-1, MUM-2, MUM-3, PAP. p53, PBF, PRAME, PSA, PSMA, RAGE-1, RNF43, RU1, RU2AS, SART-1, SART-2, SART-3, SAGE-1, SCRN 1, SOX2, SOX10, STEAPI, suivivin, tyrosinase, telomerase, TGFβ RII, TRAG-3, TRP-1, TRP-2, hTERT, WTi, and/or a neoantigen.

In some embodiments, the disclosure provides a composition comprising a drug-loaded nanoparticle that comprises one or more therapeutic agents. In some embodiments, the drug-loaded nanoparticle comprises 1, 2, 3, 4, 5, or more than 5 different therapeutic agents. In some embodiments, the drug-loaded nanoparticle comprises 1-5, 1-10, 1-15, 1-20, 1-25, 1-30, 2-10, 2-15, 2-20, 2-25, 3-10, 3-15, 3-20, 3-25, 4-10, 4-15, 4-20, 4-25, 5-10, 5-15, 5-20, or 5-25 different therapeutic agents. In some embodiments, the drug-loaded nanoparticle comprises at least one active agent that is a therapeutic agent. The therapeutic agent may be any physiologically or pharmacologically active substance that may produce a desired biological effect in a targeted site in a subject. The therapeutic agent may be, without limitation, an anti-cancer/anti-neoplastic agent (e.g., a chemotherapeutic agent, a radioisotope, an antineoplastic agent, a cytotoxic agent, a cytostatic agent, and a immunotherapeutic agent), an anti-angiogenic agent, an antibiotic, an anti-infective agent (e.g., an anti-microbial, anti-viral, anti-fungal, anti-parasitic, anti-protozoan, or anti-helminth agent), a steroid, a hormone, a cytokine, an enzyme, a cofactor, an antioxidant, a radical scavenger, a hormone, an immunomodulating agent (e.g., an immunosuppressive agent or an immunostimulant), an anti-inflammatory agent, a steroid, a vasodilator, an angiotensin converting enzyme inhibitor, an angiotensin receptor antagonist, a platelet aggregation inhibitor, an anticoagulant, or an anti-lipidemic agent, or a derivative or analog thereof.

In some embodiments, at least one therapeutic agent contained in a provided drug-loaded nanoparticle is an anti-cancer/anti-neoplastic agent. In some embodiments, the at least one anti-cancer agent is a chemotherapeutic agent. In further embodiments, the chemotherapeutic agent is an alkylating agent, an anti-metabolite, an anti-tumor antibiotic, a topoisomerase inhibitor, a mitotic inhibitor, a plant alkaloid, a microtubule inhibitor, a DNA linking agent, an immunotherapeutic agent, or a differentiating agent.

Chemotherapeutics agents that can be contained in the provided drug-loaded nanoparticles include without limitation, an alkylating agent (e.g., busulfan, carmustine), an anti-metabolite (e.g., 5-fluoro uracil, gemcitabine, methotrexate), an anti-tumor antibiotic (e.g. dactinomycin, doxorubicin, epirubicin), a topoisomerase inhibitor (e.g. topotecan, irinotecan), a mitotic inhibitor (e.g., paclitaxel, ixabepilone, vinblastine, estramustine), a plant alkaloid or a microtubule inhibitor (e.g. docetaxel, irinotecan, etoposide), a DNA linking agent (e.g., carboplatin, cisplatin, oxaliplatin), an immunotherapeutic agent (e.g, rituximab, alemtuzumab, lenalidomide), and a differentiating agent (e.g. tretinoin, bexarotene), cisplatin, doxorubicin, etoposide, irinotecan, topotecan, paclitaxel, docetaxel, tamoxifen, 5-fluorouracil, methotrexate, temozolomide, cyclophosphamide, gefitinib, erlotinib hydrochloride, actinomycin, all-trans retinoic acid, azacitidine, azathioprine, imatinib mesylate, cytarabine, gemcitabine, uracil mustard, chlormethine, ifosfamide, chlorambucil, pipobroman, triethylenemelamine, busulfan, carmustine, lomustine, streptozocin, dacarbazine, floxuridine, 6-mercaptopurine, 6-thioguanine, fludarabine phosphate, oxaliplatin, bortezomib, doxifluridine, epothilone, mechlorethamine, pemetrexed, tioguanine, valrubicin, pentostatin, vinblastine, vincristine, vindesine, bleomycin, dactinomycin, daunorubicin, epirubicin, idarubicin, deoxycofornycin, mitomycin-C, L-asparaginase, teniposide, testolactone, estramustine, carboplatin, hydroxyurea, amsacrine, procarbazine, mitotane, mitoxantrone, vinorelbine, anastrazole, letrozole, capecitabine, raloxifene, xeloda, vinorelbine, cetuximab, N,N′N′-triethylenethiophosphoramide, altretamine, trastuzumab, fulvestrant, and exemestane, and any combination thereof.

In some embodiments, at least one therapeutic agent contained in a provided drug-loaded nanoparticle is an anti-angiogenic agent. Anti-angiogenic agents that can be contained in the drug-loaded nanoparticles include without limitation, a VEGF inhibitor, bevacizumab, thalidomide, itraconazole, carboxyamidotriazole, TNP-470. IFN-α, IL-12, platelet factor-4, suramin, thrombospondin, angiostatin, endostatin, 2-methoxyestradiol, tecogalan, prolactin, linomide, ranibizumab, sorafenib, sunitinib, pazopanib, and everolimus.

In some embodiments, at least one therapeutic agent contained in a provided drug-loaded nanoparticle is a steroid. Steroids that can be contained in the provided drug-loaded nanoparticles include without limitation, a corticosteroid such as cortisone, dexamethasone, hydrocortisone, methylprednisolone, prednisolone, or prednisone.

In some embodiments, at least one therapeutic agent contained in a provided drug-loaded nanoparticle is an immunosuppressant. Immunosuppressants that can be contained in the provided drug-loaded nanoparticles include without limitation, azathioprine, chlorambucil, cyclophosphamide, cyclosporine, daclizumab, infliximab, methotrexate, and tacrolimus.

In some embodiments, at least one therapeutic agent contained in a provided drug-loaded nanoparticle is an agent useful in treating or preventing and infectious disease.

In some embodiments, at least one therapeutic agent contained in a provided drug-loaded nanoparticle is an anti-microbial agent. Anti-microbial agents that can be contained in the provided drug-loaded nanoparticles include without limitation, an aminoglycoside (e.g., gentamicin, neomycin, and streptomycin), a penicillin (e.g., amoxicillin and ampicillin), and a macrolide (e.g., erythromycin).

In some embodiments, at least one therapeutic agent contained in a provided drug-loaded nanoparticle is an anti-fungal agent. Anti-fungal agents that can be contained in the provided drug-loaded nanoparticles include without limitation, a polyene anti-fungal agent (e.g., amphotericin B and candicidin), an imidazole anti-fungal agent (e.g., bifonazole, clotrimazole, and econazole), a triazole anti-fungal agent (e.g., albaconazole, efinaconazole, and fluconazole), a thiazole anti-fungal agent (e.g., abafungin), an allylamine anti-fungal agent (e.g., amorolfin, butenafine, and naftifine), and an echinocandin (e.g., anidulafungin and caspofungin).

In some embodiments, at least one therapeutic agent contained in a provided drug-loaded nanoparticle is an anti-inflammatory agent. Anti-inflammatory agents that can be contained in the provided drug-loaded nanoparticles include without limitation, aspirin, choline salicylates, celecoxib, diclofenac potassium, diclofenac sodium, diclofenac sodium with misoprostol, diflunisal, etodolac, fenoprofen, flurbiprofen, ibuprofen, ketoprofen, meclofenamate sodium, mefenamic acid, nabumetone, naproxen, naproxen sodium, oxaprozin, piroxican, rofecoxib, salsalate, sodium salicylate, sulindac, tolmetin sodium, and valdecoxib.

### Therapeutic Uses

In some embodiments, the disclosure provides a method of inducing an immune response against one or more antigen(s) in a subject that comprises administering an immunogenic amount of a drug-loaded nanoparticle provided herein comprising the one or more antigens and/or nucleic acid(s) encoding the one or more antigens (e.g., 1, 2, 3, 4, 5, or more than 5 different antigens, or 1-5, 1-10, 1-15. 1-20, 1-25, 1-30, 2-10, 2-15, 2-20, 2-25, 3-10, 3-15, 3-20, 3-25, 4-10, 4-15. 4-20, 4-25, 5-10, 5-15, 5-20, or 5-25 different antigens). In further embodiments, the one or more antigen(s) contained in the drug-loaded nanoparticle is a protein (e.g., a glycoprotein) or peptide. In some embodiments, the one or more antigens is derived from or corresponds to an antigen from an infectious agent or a cancer. In further embodiments, the one or more antigen(s) is a polypeptide(s) and/or a fragment(s) thereof, and/or a nucleic acid(s) and/or fragment(s) thereof that is derived from or corresponds to a protein or peptide of an infectious agent such as a virus, bacteria, fungus, protozoan, and/or a parasite. In some embodiments, the one or more antigen(s) is a polypeptide(s) and/or a fragment(s) thereof, and/or a nucleic acid(s) and/or fragment(s) thereof that is derived from or corresponds to a protein or peptide expressed by a cancer. In some embodiments, the subject is a human. In some embodiments, the subject is immunocompromised or is predisposed to be immunocompromised (e.g., an older or elderly subject, e.g., over 50, 55, 60, 65, 70, 75, or 80 years of age).

As used herein “subject” or “individual” or “animal” or “patient” or “mammal,” refers to any subject, particularly a mammalian subject, for whom diagnosis, prognosis, or therapy is desired. Mammalian subjects include, but are not limited to, humans, domestic animals, farm animals, zoo animals, sport animals, pet animals such as dogs, cats, guinea pigs, rabbits, rats, mice, horses, cattle, cows; primates such as apes, monkeys, orangutans, and chimpanzees; canids such as dogs and wolves; felids such as cats, lions, and tigers; equids such as horses, donkeys, and zebras; bears, food animals such as cows, pigs, and sheep; ungulates such as deer and giraffes; rodents such as mice, rats, hamsters and guinea pigs; and so on. In certain embodiments, the mammal is a human subject. In other embodiments, a subject is a human patient. In a particular embodiment, a subject is a human patient in need of treatment.

Any techniques known to one of ordinary skill in the art may be used to determine if an immune response is elicited following administration of a vaccine provided herein. Successful immunization may further be determined in a number of additional ways known to the skilled person including, but not limited to, hemagglutination inhibition (HAI) and serum neutralization inhibition assays to detect functional antibodies; challenge studies, in which vaccinated subjects are challenged with the associated pathogen to determine the efficacy of the vaccination; and the use of fluorescence activated cell sorting (FACS) to determine the population of cells that express a specific cell surface marker, e.g. in the identification of activated or memory lymphocytes. Also, vaccine efficacy in stimulating a humoral immune response can be assessed by ELISA detection of antigen-specific antibody levels in the serum of immunized subjects. A skilled person may also determine if immunization with a composition of the invention elicited a humoral (or antibody mediated) response using other known methods. See, for example, Current Protocols in Immunology Coligan et al., ed. (Wiley Interscience, 2007). Techniques known in the art can likewise routinely be applied to determine if an immune response to an antigen vaccine provided herein is of comparable magnitude to for example, another vaccine or in the case of a multiple vaccine antigen each antigen as a single antigen vaccine or another vaccine. For example, enzyme-linked immune absorbent spot (ELISPOT) (e.g., for secretion of IFNγ) may determine the magnitude of the immune response. In some cases, the ELISPOT may detect rodent, non-human primate or human peptides.

In some embodiments, the disclosure provides a method of inducing an immune response to an infectious agent in a subject that comprises administering an immunogenic amount of drug-loaded nanoparticles provided herein that comprises one or more antigens (e.g., 1, 2, 3, 4, 5, or more than 5 different antigens, or 1-5, 1-10, 1-15, 1-20, 1-25, 1-30, 2-10, 2-15, 2-20, 2-25, 3-10, 3-15, 3-20, 3-25, 4-10, 4-15, 4-20, 4-25, 5-10, 5-15, 5-20, or 5-25 different antigens) that is derived from or corresponds to an antigen(s) from an infectious agent. In some embodiments, the infectious agent is a virus, bacteria, fungus, protozoan, and/or a parasite. In some embodiments, the subject is a human. In some embodiments, the subject is immunocompromised or is predisposed to be immunocompromised (e.g., an older or elderly subject, e.g., over 50, 55, 60, 65, 70. 75, or 80 years of age), or a subject with underlying medical condition(s) such as diabetes and cancer).

In some embodiments, the disclosure provides a method of inducing an immune response to a cancer in a subject that comprises administering an immunogenic amount of a drug-loaded nanoparticle provided herein that comprises one or more antigens (e.g., 1, 2, 3, 4, 5, or more than 5 different antigens, or 1-5, 1-10, 1-15, 1-20, 1-25, 1-30, 2-10, 2-15, 2-20, 2-25, 3-10, 3-15, 3-20, 3-25, 4-10. 4-15, 4-20, 4-25, 5-10, 5-15. 5-20, or 5-25 different antigens) that is derived from or corresponds to an antigen(s) expressed by a cancer. In some embodiments, one or more of the antigens is expressed by a cancer in the subjection. In some embodiments, the subject is a human. In some embodiments, the subject is immunocompromised or is predisposed to be immunocompromised (e.g., an older or elderly subject, e.g., over 50, 55, 60, 65, 70, 75, or 80 years of age), or a subject with underlying medical condition(s) such as diabetes and cancer).

In some embodiments, provided drug-loaded nanoparticles comprises and/or is administered in combination with a composition that is an adjuvant. As used herein, “adjuvant” means an agent that does not constitute a specific antigen, but modifies (Th1/Th2), boosts the strength and longevity of an immune response, and/or broadens the immune response to a concomitantly administered antigen. Adjuvants that can be contained in and/or administered in combination with the provided drug-loaded nanoparticles include without limitation alum (e.g., aluminum phosphate, aluminum sulfate or aluminum hydroxide), calcium phosphate, liposomes, oil-in-water emulsions such as MF59 (4.3% w/v squalene, 0.5% w/v polysorbate 80 (Tween®80), 0.5% w/v sorbitan trioleate (Span 85)), water-in-oil emulsions such as Montanide, inulin, algammaulin, monophosphoryl lipid A (MPL), resiquimod, muramyl dipeptide (MDP), N-glycolyl dipeptide (GMDP), polylC, CpG oligonucleotide, aluminum hydroxide with MPL, and poly(D,L-lactide-co-glycolide) (PLG) microparticles or nanoparticles.

In additional embodiments, the disclosure provides a method of vaccinating a subject against one or more antigens that comprises administering to the subject an effective amount of drug-loaded nanoparticles provided herein that comprise the one or more antigens. In some embodiments, the administered drug-loaded nanoparticles comprise 1, 2, 3, 4, 5, or more than 5 different antigens. In some embodiments, the administered drug-loaded nanoparticles contain 1-5, 1-10, 1-15, 1-20, 1-25, 1-30, 2-10, 2-15, 2-20, 2-25, 3-10, 3-15, 3-20, 3-25, 4-10, 4-15, 4-20, 4-25, 5-10, 5-15, 5-20, or 5-25 different antigens. In some embodiments, the disclosure provides a method of vaccinating a subject against an infectious agent. In some embodiments, the disclosure provides a method of vaccinating a subject against a cancer. In some embodiments, the subject is a human. In some embodiments, the subject is immunocompromised (e.g., an old or elderly subject, e.g., over 50, 55, 60, 65, 70, 75, or 80 years of age), or a subject with underlying medical condition(s) known to be immunocompromised and susceptible to infection).

In some embodiments, the disclosure provides a method of vaccinating a subject against an infectious agent that comprises administering to the subject an effective amount of a drug-loaded nanoparticle provided herein that contains one or more different antigens derived from or corresponding to an infectious agent. In some embodiments, the administered drug-loaded nanoparticles comprise 1, 2, 3, 4, 5, or more than 5 different antigens derived from or corresponding to the infectious agent. In some embodiments, the administered drug-loaded nanoparticles contain 1-5, 1-10, 1-15, 1-20, 1-25, 1-30, 2-10, 2-15, 2-20, 2-25, 3-10, 3-15, 3-20, 3-25, 4-10, 4-15, 4-20, 4-25, 5-10, 5-15, 5-20, or 5-25 different antigens derived from or corresponding to the infectious agent. In some embodiments, the subject is a human. In some embodiments, the subject is immunocompromised (e.g., an old or elderly subject, e.g., over 50, 55, 60, 65, 70, 75, or 80 years of age), or a subject with underlying medical condition(s) known to be immunocompromised and susceptible to infection).

In some embodiments, the disclosure provides a method of vaccinating a subject against a viral infectious agent that comprises administering to the subject an effective amount of a drug-loaded nanoparticle provided herein that contains one or more different antigens derived from or corresponding to the viral infectious agent. In some embodiments, the administered drug-loaded nanoparticles comprise 1, 2, 3, 4, 5, or more than 5 different antigens derived from or corresponding to the viral infectious agent. In some embodiments, the administered drug-loaded nanoparticles contain 1-5, 1-10, 1-15, 1-20, 1-25, 1-30, 2-10, 2-15, 2-20, 2-25, 3-10, 3-15, 3-20, 3-25, 4-10, 4-15, 4-20, 4-25, 5-10, 5-15, 5-20, or 5-25 different antigens derived from or corresponding to the viral infectious agent.

Viruses, or parts thereof, useful as antigens and for which a corresponding vaccination can be accomplished according to the claimed methods include, without limitation, poxvirus, monkeypoxvirus, cowpoxvirus, vaccinia virus, pseudocowpox virus, human herpes virus (e.g., VZV, HSV-1, HAV-6, HSV-IL and CMV, Epstein Barr virus), cytomegalovirus, human adenovirus A-F, polyomavirus, human papillomavirus (HPV), parvovirus, hepatitis A virus, hepatitis B virus, hepatitis C virus, hepatitis D virus, human immunodeficiency virus (HIV), orthoreovirus, rotavirus, ebola virus, parainfluenza virus, influenza virus (e.g. H5N1 influenza virus, influenza A virus, influenza B virus, influenza C virus), measles virus, mumps virus, rubella virus, pneumovirus, severe acute respiratory syndrome virus, human respiratory syncytial virus, rabies virus, California encephalitis virus, Japanese encephalitis virus, arboviral encephalitis virus, JC virus, echovirus, coxsackie virus, HTLV virus, molluscum virus, poliovirus, rabies virus, Hantaan virus, lymphocytic choriomeningitis virus, coronavirus, enterovirus, rhinovirus, poliovirus, norovirus, flaviviruses, dengue virus, West Nile virus, yellow fever virus and varicella.

In some embodiments, the disclosure provides a method of vaccinating a subject against a bacterial infectious agent that comprises administering to the subject an effective amount of a drug-loaded nanoparticle provided herein that contains one or more different antigens derived from or corresponding to the bacterial infectious agent. In some embodiments, the administered drug-loaded nanoparticles comprise 1, 2, 3, 4, 5, or more than 5 different antigens derived from or corresponding to the bacterial infectious agent. In some embodiments, the administered drug-loaded nanoparticles contain 1-5, 1-10, 1-15, 1-20, 1-25, 1-30, 2-10, 2-15, 2-20, 2-25, 3-10, 3-15, 3-20, 3-25, 4-10, 4-15, 4-20, 4-25, 5-10, 5-15, 5-20, or 5-25 different antigens derived from or corresponding to the bacterial infectious agent.

Bacteria, or parts thereof, useful as antigens and for which a corresponding vaccination can be accomplished according to the claimed methods include, without limitation, anthrax (Bacillus anthracis), Brucella, Bordetella pertussis, Candida, streptococcal bacteria (e.g., pyogenes, agalactiae, pneumoniae), chlamydia (e.g., Chlamydia pneumoniae, Chlamydia psittaci), Cholera, Clostridium botulinum, Coccidioides immitis, Cryptococcus, Diphtheria, Escherichia coli O157: H7, Enterohemorrhagic Escherichia coli, Enterotoxigenic Escherichia coli, Haemophilus influenzae, Helicobacter pylori, legionella, leptospira, Listeria, Meningococcus, Listeria monocytogenes Mycoplasma pneumoniae, Mycobacterium (tuberculosis), Bordetella pertussis, salmonella, bacilli, shigella, Staphylococcus, rickettsia bacteria, mycobacteria, staphylococci, streptococci, pneumonococci, meningococci and conococci, klebsiella, proteus, serratia, pseudomonasand Yersinia enterocolitica.

In some embodiments, the disclosure provides a method of vaccinating a subject against a fungal infectious agent that comprises administering to the subject an effective amount of a drug-loaded nanoparticle provided herein that contains one or more different antigens derived from or corresponding to the fungal infectious agent. In some embodiments, the administered drug-loaded nanoparticles comprise 1, 2, 3, 4, 5, or more than 5 different antigens derived from or corresponding to the fungal infectious agent. In some embodiments, the administered drug-loaded nanoparticles contain 1-5, 1-10, 1-15, 1-20, 1-25, 1-30, 2-10, 2-15, 2-20, 2-25, 3-10, 3-15, 3-20, 3-25, 4-10, 4-15, 4-20, 4-25, 5-10, 5-15, 5-20, or 5-25 different antigens derived from or corresponding to the fungal infectious agent.

Fungi, or parts of thereof useful as antigens in the invention include, without limitation, Candida (albicans, krusei, glabrata, tropicalis, etc.), Ciyptococcus (Cryptococcus neoformans), Aspergillus (fumigatus, Niger, etc.), Genus Mucorales (Mucor, absidia, rhizopus), Sporothrix schenkii, Blastomyces dermatitidis, Paracoccidioides brasiliensis, Coccidioides (Coccidioides immitis) and Histoplasma capsulatum.

In some embodiments, the disclosure provides a method of vaccinating a subject against a parasitic, protozoan, or helminth infectious agent that comprises administering to the subject an effective amount of a drug-loaded nanoparticle provided herein that contains one or more different antigens derived from or corresponding to the parasitic, protozoan, or helminth infectious agent. In some embodiments, the administered drug-loaded nanoparticles comprise 1, 2, 3, 4, 5, or more than 5 different antigens derived from or corresponding to the parasitic, protozoan, or helminth infectious agent. In some embodiments, the administered drug-loaded nanoparticles contain 1-5, 1-10. 1-15, 1-20, 1-25, 1-30, 2-10, 2-15, 2-20, 2-25, 3-10, 3-15, 3-20, 3-25, 4-10, 4-15, 4-20, 4-25, 5-10, 5-15, 5-20, or 5-25 different antigens derived from or corresponding to the parasitic, protozoan, or helminth infectious agent.

Parasites, or parts thereof, useful as antigens and for which a corresponding vaccination can be accomplished according to the claimed methods include, without limitation, Entamoeba histolytica, Balantidium coli, Naegleria fowleri, Acanthamoeba sp., Giardia lambia, Cryptosporidium sp., Pneumocystis carinii, Plasmodium (Plasmodium falciparum, Plasmodium malariae, Plasmodium vivax, Plasmodium ovale or Plasmodium knowlesi), Plasmodium vivax, Babesia microti, Trypanosoma brucei, Trypanosoma cruzi, Leishmania donovani, Toxoplasma gondii, and Nippostrongylus brasiliensis.

Protozoans or parts thereof, useful as antigens and for which a corresponding vaccination can be accomplished according to the claimed methods include, without limitation, Sarcodina (e.g., Entamoeba), Mastigophora (e.g., Giardia), Ciliophora (e.g., Balantidium), and Sporozoa (e.g., Plasmodium falciparum, Cryptosporidium).

Helminths or parts thereof, useful as antigens and for which a corresponding vaccination can be accomplished according to the claimed methods include, without limitation, Platyhelminths (e.g., trematodes, cestodes), Acanthocephalins, and Nematodes.

In some embodiments, the provided drug-loaded nanoparticles comprises antigens that are derived from or correspond to antigens expressed during different lifecycle stages of an infectious agent. For example, in some embodiments, the drug-loaded nanoparticles contain antigens derived from or corresponding to antigens expressed during two or more of the sporozoite stage, blood stage, liver stage, or sexual stage of the malaria plasmodium parasite. In further embodiments the drug-loaded nanoparticles contain 1, 2, 3, 4 or more antigens corresponding to the CSP and PfSSP2 sporozoite proteins, the LSA-1 liver stage protein, the MSA-1, MSP-1, SERA, and AMA-1 blood stage proteins, and the Pfs25 sexual stage protein of plasmodium. In some embodiments, the provided drug-loaded nanoparticles comprises antigens that are derived from or correspond to plasmodium CSP, AMA1, and MSP1.

In some embodiments, the drug-loaded nanoparticles contain antigens derived from or corresponding to antigens expressed during two or more phases of an infectious disease. For example, in some embodiments, the drug-loaded nanoparticles contain antigens derived from or corresponding to antigens expressed by M. tuberculosis during 2 or more of the infectious phase, latent phase, and reactivation phase of M. tuberculosis infection

In some embodiments, the disclosure provides a drug-loaded nanoparticle that has use as a cancer vaccine. A “cancer vaccine” is an immunogenic composition intended to elicit an immune response against one or more particular antigens in the subject to which the cancer vaccine is administered. A cancer vaccine typically contains a tumor antigen which is able to induce or stimulate an immune response against the tumor antigen. A “tumor antigen” is an antigen that is present on the surface of a target tumor. A tumor antigen may be a molecule which is not expressed by a non-tumor cell or may be, for example, a neoantigen or an altered version of a molecule expressed by a non-tumor cell (e.g., a protein that is misfolded, truncated, or otherwise mutated).

In some embodiments, the a drug-loaded nanoparticle provided disclosure provides a method of vaccinating a subject against a cancer that comprises administering to the subject an effective amount of a drug-loaded nanoparticle provided herein that contains one or more different tumor antigen(s) derived from or corresponding to an antigen expressed by a cancer. In some embodiments, the administered drug-loaded nanoparticle comprises 1, 2, 3, 4, 5, or more than 5 different antigens derived from or corresponding to an antigen expressed by a cancer cell. In some embodiments, the administered drug-loaded nanoparticles contain 1-5, 1-10, 1-15, 1-20, 1-25, 1-30, 2-10, 2-15, 2-20, 2-25, 3-10, 3-15, 3-20, 3-25, 4-10, 4-15, 4-20, 4-25, 5-10, 5-15, 5-20, or 5-25 different antigens derived from or corresponding to an antigen expressed by a cancer. In some embodiments, the administered drug-loaded nanoparticles contain 1-5, 1-10, 1-15, 1-20, 1-25, 1-30, 2-10, 2-15, 2-20, 2-25, 3-10, 3-15, 3-20, 3-25, 4-10, 4-15, 4-20, 4-25, 5-10, 5-15, 5-20, or 5-25 different TAA antigens derived from or corresponding to an antigen expressed by a cancer. In some embodiments, the administered drug-loaded nanoparticles contain 1-5, 1-10, 1-15, 1-20, 1-25, 1-30, 2-10, 2-15, 2-20, 2-25, 3-10, 3-15, 3-20, 3-25, 4-10, 4-15, 4-20, 4-25, 5-10, 5-15, 5-20, or 5-25 different TSA antigens derived from or corresponding to an antigen expressed by a cancer. In some embodiments, the subject is a human. In some embodiments, the subject is immunocompromised (e.g., an old or elderly subject, e.g., over 50, 55, 60, 65, 70, 75, or 80 years of age).

The terms “cancer,” and “tumor” are used herein to refer to cells which exhibit autonomous, unregulated growth, such that the cells exhibit an aberrant growth phenotype characterized by a significant loss of control over cell proliferation. Cells of interest for detection, analysis, and/or treatment in the context of the invention include cancer cells (e.g., cancer cells from an individual with cancer), malignant cancer cells, pre-metastatic cancer cells, metastatic cancer cells, and non-metastatic cancer cells. Cancers of virtually every tissue are known. Many types of cancers are known to those of skill in the art, including solid tumors such as carcinomas, sarcomas, glioblastomas, melanomas, lymphomas, and myelomas, and circulating cancers such as leukemias. Cancer includes any form of cancer, including but not limited to, solid tumor cancers (e.g., lung, prostate, breast, gastric, bladder, colon, ovarian, pancreas, kidney, liver, glioblastoma, medulloblastoma, leiomyosarcoma, head & neck squamous cell carcinomas, melanomas, and neuroendocrine) and liquid cancers (e.g., hematological cancers); carcinomas; soft tissue tumors; sarcomas; teratomas; melanomas; leukemias; lymphomas; and brain cancers, including minimal residual disease, and including both primary and metastatic tumors.

In additional embodiments, the disclosure provides a method of treating or preventing a disease in a subject that comprises administering an effective amount of drug-loaded nanoparticles provided herein to a subject in need thereof. In some embodiments, the disease treated or prevented by the provided method is an infectious disease. In some embodiments, the disease treated or prevented by the provided method is cancer. In some embodiments, the disease treated or prevented by the provided method is a disorder of the immune system. In some embodiments, the subject is a human.

In some embodiments, the disclosure provides a method of treating or preventing an infectious disease in a subject that comprises administering to the subject an effective amount of a drug-loaded nanoparticle provided herein. The term “infectious disease”, as used herein, may refer for example to any communicable disease, contagious disease or transmissible disease or disorder resulting from the infection, presence and/or growth of a pathogenic biological agent. Without limitation, the infectious pathogenic agent may include for example a virus, bacteria, fungus, protozoan, parasite or helminth. In some embodiments, the subject is a human. In some embodiments, the subject is immunocompromised or is predisposed to be immunocompromised (e.g., an older or elderly subject, e.g., over 50, 55, 60, 65, 70, 75, or 80 years of age), or a subject with underlying medical condition(s) such as diabetes and cancer).

In some embodiments, the administered drug-loaded nanoparticle contains one or more antigens (e.g., 1, 2, 3, 4, 5, or more than 5 different antigens, or 1-5, 1-10, 1-15, 1-20, 1-25, 1-30, 2-10, 2-15, 2-20, 2-25, 3-10, 3-15, 3-20, 3-25, 4-10, 4-15, 4-20, 4-25, 5-10, 5-15, 5-20, or 5-25 different antigens) that is derived from or corresponds to an antigen(s) from an infectious agent associated with the infectious disease. In some embodiments, the infectious disease is caused by a viral infectious agent. In some embodiments, the infectious disease is caused by a bacterial infectious agent. In some embodiments, the infectious disease is caused by a parasitic infectious agent. In some embodiments, the infectious disease is caused by a fungal, protozoan, or helminth infectious agent.

In some embodiments, the disclosure provides a method of treating or preventing an infectious disease in a subject that comprises administering to the subject an effective amount of a drug-loaded nanoparticle that contains one or more therapeutic agents useful for treating or preventing the infectious disease. In some embodiments, the drug-loaded nanoparticle contains 2, 3, 4, 5, or more than 5 therapeutic agents, or 1-15, 1-10 or 1-5 therapeutic agents useful for treating or preventing an infectious disease. In some embodiments, the infectious disease is caused by a viral infectious agent. In some embodiments, the infectious disease is caused by a bacterial infectious agent. In some embodiments, the infectious disease is caused by a parasitic infectious agent. In some embodiments, the infectious disease is caused by a fungal, protozoan, or helminth infectious agent.

In some embodiments, the administered drug-loaded nanoparticle contains one or more antigens (e.g., 1, 2, 3, 4, 5, or more than 5 different antigens, or 1-5, 1-10, 1-15, 1-20, 1-25, 1-30, 2-10, 2-15, 2-20, 2-25, 3-10, 3-15, 3-20, 3-25, 4-10, 4-15, 4-20, 4-25, 5-10, 5-15, 5-20, or 5-25 different antigens) that is derived from or corresponds to an antigen(s) from an infectious agent associated with the infectious disease; and the drug-loaded nanoparticle further contains 2, 3, 4, 5, or more than 5 therapeutic agents, or 1-15, 1-10 or 1-5 therapeutic agents useful for treating or preventing the infectious disease. In some embodiments, the infectious disease is caused by a bacterial infectious agent. In some embodiments, the infectious disease is caused by a parasitic infectious agent. In some embodiments, the infectious disease is caused by a fungal, protozoan, or helminth infectious agent.

Non-limiting examples of infectious diseases that may be treated or prevented by the provided methods include without limitation influenza (e.g. infection by influenza virus), respiratory tract infections such as, for example, bronchiolitis and pneumonia (e.g. infection by respiratory syncytial virus), pertussis or whooping cough (e.g. infection by Bordetella pertussis), herpes disease (e.g., genital herpes, chicken pox or herpes zoster (shingles), infectious mononucleosis), tuberculosis infection (caused by Mycobacterium tuberculosis), typhoid infection or fever (caused by Salmonella typhi), anthrax (e.g. infection by Bacillus anthracis), coccidioidomycosis, and malaria (e.g., infection by Plasmodium malariae, Plasmodium falciparum, Plasmodium vivax, Plasmodium ovale or Plasmodium knowlesi), respiratory papillomatosis, shigellosis salmonella, cholera, tetanus, botulism, plague, leptospirosis, Lyme's disease, monkeypox virus infection, west nile virus infection, chikungunya virus infection, ebola virus infection, ebola hemorrhagic fever, hepatitis A, B, C, or D virus infection, poliovirus infection, dengue fever, acquired immune deficiency syndrome (AIDS) or a simian immunodeficiency virus (SIV) infection.

Examples of viruses causing infections and their associated conditions that are treatable by methods of the present disclosure include without limitation, poxvirus, monkeypoxvirus, cowpoxvirus, vaccinia virus, pseudocowpox virus, human herpes virus (e.g., VZV, HSV-1, HAV-6, HSV-II, and CMV, Epstein Barr virus), cytomegalovirus, human adenovirus A-F, polyomavirus, human papillomavirus (HPV), parvovirus, hepatitis A virus, hepatitis B virus, hepatitis C virus, hepatitis D virus, human immunodeficiency virus (HIV), orthoreovirus, rotavirus, ebola virus, parainfluenza virus, influenza virus (e.g. H5N1 influenza virus, influenza A virus, influenza B virus, influenza C virus), measles virus, mumps virus, rubella virus, pneumovirus, severe acute respiratory syndrome virus, human respiratory syncytial virus, rabies virus, California encephalitis virus. Japanese encephalitis virus, arboviral encephalitis virus, JC virus, echovirus, coxsackie virus, HTLV virus, molluscum virus, poliovirus, rabies virus, Hantaan virus, lymphocytic choriomeningitis virus, coronavirus, enterovirus, rhinovirus, poliovirus, norovirus, flaviviruses, dengue virus, West Nile virus, yellow fever virus and varicella.

Examples of bacteria causing infections and their associated conditions that are treatable by methods of the present disclosure include without limitation, anthrax (Bacillus anthracis), Brucella, Bordetella pertussis, Candida, streptococcal bacteria (e.g., pyogenes, agalactiae, pneumoniae), chlamydia (e.g., Chlamydia pneumoniae, Chlamydia psittaci), Cholera, Clostridium botulinum, Coccidioides immitis, Cryptococcus, Diphtheria, Escherichia coli O157: H7, Enterohemorrhagic Escherichia coli, Enterotoxigenic Escherichia coli, Haemophilus influenzae, Helicobacter pylori, legionella, leptospira, Listeria, Meningococcus, Listeria monocytogenes Mycoplasma pneumoniae, Mycobacterium (tuberculosis), Bordetella pertussis, salmonella, bacilli, shigella, Staphylococcus, rickettsia bacteria, mycobacteria, staphylococci, streptococci, pneumonococci, meningococci and conococci, klebsiella, proteus, serratia, pseudomonas and, and Yersinia enterocolitica.

Examples of fungi causing infections and their associated conditions that are treatable by methods of the present disclosure include without limitation, Candida (albicans, krusei, glabrata, tropicalis, etc.), Cryptococcus (Cryptococcus neoformans), Aspergillus (fumigatus, Niger, etc.), Genus Mucorales (Mucor, absidia, rhizopus), Sporothrix schenkii, Blastomyces dermatitidis, Paracoccidioides brasiliensis, Coccidioides (Coccidioides immitis) and Histoplasma capsulatum.

Examples of parasites causing infections and their associated conditions that are treatable by methods of the present disclosure include without limitation, Entamoeba histolytica, Balantidium coli, Naegleria fowleri, Acanthamoeba sp., Giardia lambia, Cryptosporidium sp., Pneumocystis carinii, Plasmodium (Plasmodium falciparum, Plasmodium malariae, Plasmodium vivax, Plasmodium ovale or Plasmodium knowlesi), Plasmodium vivax, Babesia microti, Trypanosoma brucei, Trypanosoma cruzi, Leishmania donovani, Toxoplasma gondii, and Nippostrongylus brasiliensis.

Examples of protozoans causing infections and their associated conditions that are treatable by methods of the present disclosure include without limitation, Protozoans or parts thereof, useful as antigens and for which a corresponding vaccination can be accomplished according to the claimed methods include, without limitation, Sarcodina (e.g., Entamoeba), Mastigophora (e.g., Giardia), Ciliophora (e.g., Balantidium), and Sporozoa (e.g., Plasmodium falciparum, Cryptosporidium).

Examples of helminths causing infections and their associated conditions that are treatable by methods of the present disclosure include without limitation, Helminths or parts thereof, useful as antigens and for which a corresponding vaccination can be accomplished according to the claimed methods include, without limitation, Examples of helminths include Platyhelminths (e.g., trematodes, cestodes), Acanthocephalins, and Nematodes.

In additional embodiments, the disclosure provides a method of treating or preventing cancer in a subject that comprises administering to the subject an effective amount of a drug-loaded nanoparticle provided herein that contains one or more different tumor antigen(s) derived from or corresponding to an antigen expressed by a cancer. In some embodiments, the administered drug-loaded nanoparticle comprise 1, 2, 3, 4, 5, or more than 5 different antigens derived from or corresponding to an antigen expressed by a cancer cell. In some embodiments, the administered drug-loaded nanoparticle contains 1-5, 1-10, 1-15, 1-20, 1-25, 1-30, 2-10, 2-15, 2-20, 2-25, 3-10, 3-15, 3-20, 3-25, 4-10, 4-15, 4-20, 4-25, 5-10, 5-15, 5-20, or 5-25 different antigens derived from or corresponding to an antigen expressed by a cancer. In some embodiments, the subject is a human. In some embodiments, the subject is immunocompromised or is predisposed to be immunocompromised (e.g., an older or elderly subject, e.g., over 50, 55, 60, 65, 70, 75, or 80 years of age), or a subject with underlying medical condition(s) such as diabetes and cancer).

In some embodiments, the disclosure provides a method of treating or preventing a cancer in a subject that comprises administering to the subject an effective amount of a drug-loaded nanoparticle that contains one or more therapeutic agents useful for treating or preventing cancer. In some embodiments, the drug-loaded nanoparticle contains 2, 3, 4, 5, or more than 5 therapeutic agents, or 1-15, 1-10 or 1-5 therapeutic agents useful for treating or preventing cancer. In some embodiments, the infectious disease is caused by a viral infectious agent. In some embodiments, the infectious disease is caused by a bacterial infectious agent. In some embodiments, the infectious disease is caused by a parasitic infectious agent. In some embodiments, the infectious disease is caused by a fungal, protozoan, or helminth infectious agent.

In some embodiments, the administered drug-loaded nanoparticle contains at least one anti-cancer agent. In some embodiments, the anti-cancer agent is a chemotherapeutic agent. Chemotherapeutics agents that can be contained in an administered drug-loaded nanoparticle include without limitation, an alkylating agent (e.g., busulfan, carmustine), an anti-metabolite (e.g., 5-fluoro uracil, gemcitabine, methotrexate), an anti-tumor antibiotic (e.g. dactinomycin, doxorubicin, epirubicin), a topoisomerase inhibitor (e.g. topotecan, irinotecan), a mitotic inhibitor (e.g., paclitaxel, ixabepilone, vinblastine, estramustine), a plant alkaloid or a microtubule inhibitor (e.g. docetaxel, irinotecan, etoposide), a DNA linking agent (e.g., carboplatin, cisplatin, oxaliplatin), an immunotherapeutic agent (e.g., rituximab, alemtuzumab, lenalidomide), and a differentiating agent (e.g. tretinoin, bexarotene), cisplatin, doxorubicin, etoposide, irinotecan, topotecan, paclitaxel, docetaxel, tamoxifen, 5-fluorouracil, methotrexate, temozolomide, cyclophosphamide, gefitinib, erlotinib hydrochloride, actinomycin, all-trans retinoic acid, azacitidine, azathioprine, imatinib mesylate, cytarabine, gemcitabine, uracil mustard, chlormethine, ifosfamide, chlorambucil, pipobroman, triethylenemelamine, busulfan, carmustine, lomustine, streptozocin, dacarbazine, floxuridine, 6-mercaptopurine, 6-thioguanine, fludarabine phosphate, oxaliplatin, bortezomib, doxifluridine, epothilone, mechlorethamine, pemetrexed, tioguanine, valrubicin, pentostatin, vinblastine, vincristine, vindesine, bleomycin, dactinomycin, daunorubicin, epirubicin, idarubicin, deoxycofornycin, mitomycin-C, L-asparaginase, teniposide, testolactone, estramustine, carboplatin, hydroxyurea, amsacrine, procarbazine, mitotane, mitoxantrone, vinorelbine, anastrazole, letrozole, capecitabine, raloxifene, xeloda, vinorelbine, cetuximab, N,N′N′-triethylenethiophosphoramide, altretamine, trastuzumab, fulvestrant, exemestane, and any combination thereof.

In some embodiments, a therapeutic agent contained in an administered drug-loaded nanoparticle is an anti-angiogenic agent. Anti-angiogenic agents that can be contained in the administered drug-loaded nanoparticles include without limitation, a VEGF inhibitor, bevacizumab, thalidomide, itraconazole, carboxyamidotriazole, TNP-470, IFN-α, IL-12, platelet factor-4, suramin, thrombospondin, angiostatin, endostatin, 2-methoxyestradiol, tecogalan, prolactin, linomide, ranibizumab, sorafenib, sunitinib, pazopanib, and everolimus.

In some embodiments, the administered drug-loaded nanoparticle contains one or more different tumor antigen(s) derived from or corresponding to an antigen expressed by a cancer and one or more therapeutic agent. In some embodiments, the administered drug-loaded nanoparticle comprise 1, 2, 3, 4, 5, or more than 5 different antigens (e.g., 1-5, 1-10, 1-15, 1-20, 1-25, 1-30, 2-10, 2-15, 2-20, 2-25, 3-10, 3-15, 3-20, 3-25, 4-10, 4-15, 4-20, 4-25, 5-10. 5-15, 5-20, or 5-25 different antigens) derived from or corresponding to an antigen expressed by a cancer cell and a therapeutic agent. In some embodiments, the administered drug-loaded nanoparticle contains 1, 2, 3, 4, 5, or more than 5 different antigens (e.g., 1-5, 1-10, 1-15, 1-20, 1-25, 1-30, 2-10, 2-15, 2-20, 2-25, 3-10, 3-15, 3-20, 3-25, 4-10, 4-15, 4-20, 4-25, 5-10, 5-15, 5-20, or 5-25 different antigens) derived from or corresponding to an antigen expressed by a cancer cell; and contains 2, 3, 4, 5, or more than 5 therapeutic agents, or 1-15, 1-10 or 1-5 therapeutic agents useful for treating or preventing cancer. In some embodiments, the subject is a human. In some embodiments, the subject is immunocompromised or is predisposed to be immunocompromised (e.g., an older or elderly subject, e.g., over 50, 55, 60, 65, 70, 75, or 80 years of age).

In some embodiments, the disclosure provides a method of treating or preventing cancer in a subject that comprises administering to the subject an effective amount of a drug-loaded nanoparticle that contains one or more therapeutic agents useful for treating or preventing cancer. In some embodiments, the drug-loaded nanoparticle contains 2, 3, 4, 5, or more than 5 therapeutic agents, or 1-15, 1-10 or 1-5 therapeutic agents useful for treating or preventing cancer.

### Definitions

As used herein, the singular form “a”, “an”, and “the”, includes plural references unless it is expressly stated or is unambiguously clear from the context that such is not intended.

As used herein, the term “about” modifying an amount related to the invention refers to variation in the numerical quantity that can occur, for example, through routine testing and handling; through inadvertent error in such testing and handling; through differences in the manufacture, source, or purity of ingredients employed in the invention; and the like. As used herein, “about” a specific value also includes the specific value, for example, about 10% includes 10%. As used herein, when “about” is used to modify a range, both the lower limit and higher limit should be understood as preceding with the term “about”, and the lower limit and higher limit should have the same unit unless otherwise specified. For example, about 1-5 mM should be understood as about 1 mM to about 5 mM. Whether or not modified by the term “about”, the claims include equivalents of the recited quantities. In one embodiment, the term “about” means within 20% of the reported numerical value.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

Where, features or embodiments of the disclosure are described in terms of a Markush group, it is intended that the disclosure is also thereby described in terms of any individual member or subgroup of members of the Markush group.

The use of “including,” “comprising,” or “having,” “containing”, “involving”, and variations thereof herein, is meant to encompass the items listed thereafter and equivalents thereof as well as additional items.

As used herein, the terms “treatment”, “treat” and “treating,” refers to partially or completely alleviating, ameliorating, improving, relieving, delaying onset of, inhibiting progression of, reducing severity of, and/or reducing incidence of one or more symptoms or features of a particular infection, disease, disorder, and/or condition. For example, “treating” cancer may refer to inhibiting survival, growth, and/or spread of a tumor. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

As used herein, the terms “prevent”, “preventing” and “prevention” refer to prophylactic and preventative measures, wherein the object is to reduce the chances that a subject will develop the pathologic condition or disorder over a given period of time. Such a reduction may be reflected, e.g., in a delayed onset of at least one symptom of the pathologic condition or disorder in the subject The term “prophylactic” refers to a treatment administered to a subject who does not exhibit signs of a disease or exhibits only early signs for the purpose of decreasing the risk of developing pathology.

As used herein, the term “therapeutically effective amount” means an amount of an agent to be delivered (e.g., nucleic acid, drug, therapeutic agent, diagnostic agent, prophylactic agent, etc.) that is sufficient, when administered to a subject suffering from or susceptible to an infection, disease, disorder, and/or condition, to treat, improve symptoms of, diagnose, prevent, and/or delay the onset of the infection, disease, disorder, and/or condition.

Headings and subheadings are used for convenience and/or formal compliance only, do not limit the subject technology, and are not referred to in connection with the interpretation of the description of the subject technology. Features described under one heading or one subheading of the subject disclosure may be combined, in various embodiments, with features described under other headings or subheadings. Further it is not necessarily the case that all features under a single heading or a single subheading are used together in embodiments.

### EXAMPLES

### Example 1. TMC Precursor Synthesis and Characterization

TMC samples with different degrees of methylation (quaternization) are synthesized according to the method described below.

Briefly, chitosan was methylated by methyl iodide in a strong base (NaOH) solution at 60° C. for 9-24 hours to obtain TMC with different degrees of quaternization (20-50%). Here, we methylated chitosan twice to reach a high level of methylation. After the methylation reaction, the products were dissolved in NaCl solution and then purified by dialysis against the water and finally lyophilized. The purified products were then analyzed by ¹H NMR spectroscopy. The proton nuclear magnetic resonance (¹H-NMR) spectrum of TMC is shown in FIG. 2. According to the literature (Tafaghodi, et al. 2012), the signal at 3.22 ppm corresponds to the methyl group at the N,N,N-trimethylated site, the signal at 2.72 ppm corresponds to the methyl group at the N,N-demethylated site, and the signals ranging from 4.8 to 5.4 ppm are attributed to the hydrogen atom bonded to the carbon 1 of the glycoside ring. The degree of quaternization (DQ) of the final product was calculated as approximately 50.5% using the equation shown in the FIG. 2 insert. The ∫_{TM} is the integral of the trimethyl amino group (quaternary amino group) peak at 3.3 ppm and ∫_{H} is the integral of the ¹H peaks from 4.7 to 5.7 ppm.

Furthermore, the FTIR spectra of TMC and chitosan were measured using a Nexus 6700 FTIR with Diamond ATR insert. The band shown in FIG. 3 at 1,471 cm⁻¹ was attributed to angular deformation of C—H bonds of methyl groups existing in higher proportion in TMC (Xu 2013), as compared with spectrum of chitosan only which has no dominant peak on this contribution. The bands at 2,918 cm⁻¹ that appear in the FTIR spectrum of TMC were attributed to characteristic stretching of C—H bonds. The IR spectrum further establishes that we successfully obtained the trimethylation of TMC.

### Example 2. LbL Composition Using Traditional Synthesis Method

To form crosslinked TMC nanoparticles, cross-linker materials must be involved. Tripolyphosphate (TPP) is a non-toxic polyanion which can crosslink with TMC to form uniform nanoparticles. By using a cross linker, the encapsulation and loading of multiple antigens on the crosslinked TMC nanoparticles can be achieved. Significant effort was given to investigating these polymer compositions, such as the ratio between TMC and TPP. This information was important to reaching the goal of multiple protein loading and release. There were many parameters of reaction that influenced the crosslinked TMC nanoparticle formation. We executed experiments with hundreds of factors. We determined that the reaction time and composition were the most important factors among these conditions (reaction time, chemical composition, stirring speeds, etc.) as shown in the Table 1.

TABLE 1
All the parameters evaluated and tested
in the formation of LbL NP formulations.

| Component | Range (wt.-%) | Optimal/Preferred |
|---|---|---|
| TMC | 62.5-83.3% | 82.6-83.3% |
| TPP | 9.1-16.7% | 16.5-16.7% |
| PSS | 0.83-67% | 0.83% |
| HA | 16.4-0.83% | 0.83-4% |
| Variable | Range | Optimal/Preferred |
| TMC Degree of | 20-50% | 40-50% |
| Quaternization | | |
| Stir Rate | 110 rpm-2000 rpm | 700 rpm |
| Reaction time | 0-24 | 1 |
| (hours) | | |
| Ratio of TMC/TPP | 5:1-10:1 | 5:1-7:1 |
| Ratio of | 5:1:2-5:1:0.01 | 5:1:0.01 |
| TMC/TPP/PSS | | |
| Ratio of | 5:1:0.2-5:1:0.01 | 5:1:0.05-5:1:0.01 |
| TMC/TPP/HA | | |

TMC-TPP ratio: We first evaluated the nanoparticle formation in a small glass container (10 ml). 10 mg of TMC was dissolved in 5 ml of DI water to obtain 2 mg/ml concentration. Subsequently, 2 ml (1 mg/ml) of TPP solution (pH 8) was slowly added dropwise to the TMC solution (pH 6) while at stirring or non-stirring at ambient temperature, yielding a final pH of around 7 NP solution, but the ratio of TMC/TPP were selected at between 10:1-5:1. At ratio between 7:1-5:1, we observed good NP formation (Table 1).

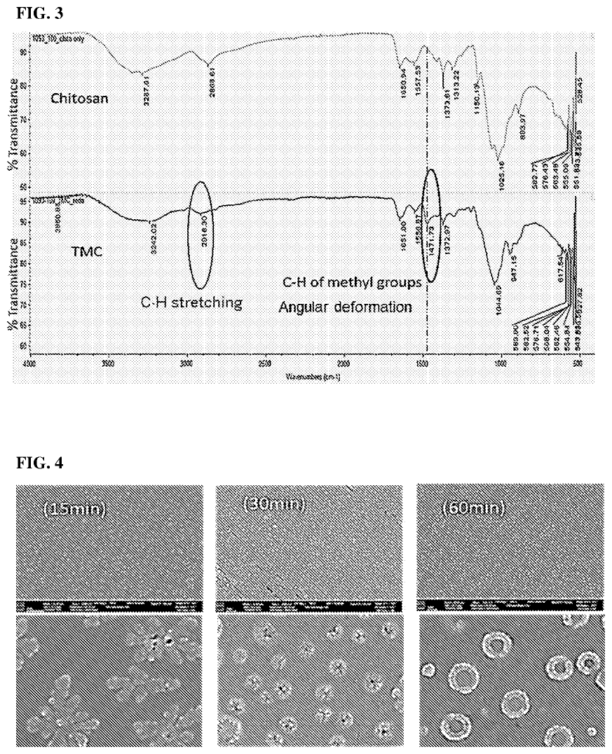

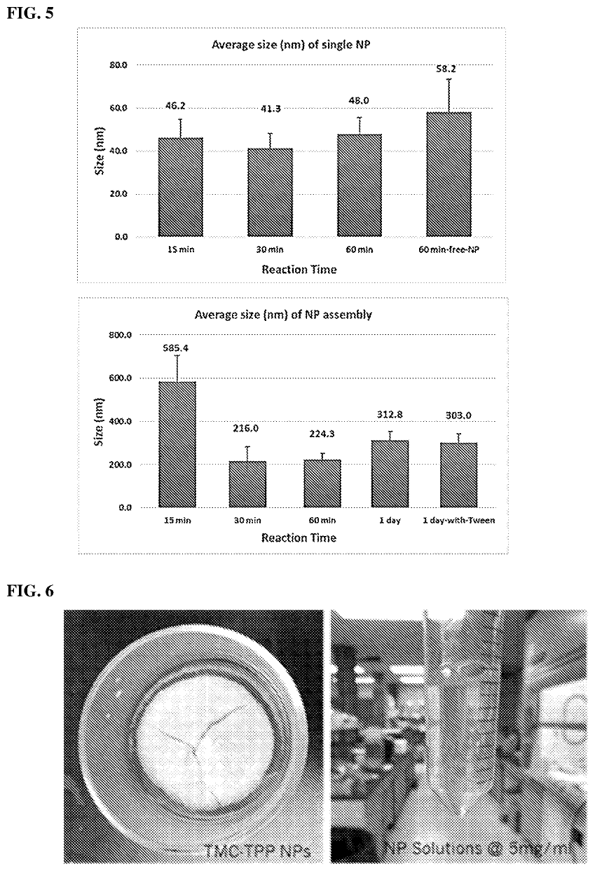

Reaction time: The LbL NP reaction was performed in a glass container. We evaluated the NP formation at different reaction times between 0 and 24 hours. After 15 min, the particles were starting to form. Between 30-60 minutes of reaction time, the nanoparticles were stable at approximately 50 nm diameter as a single nanoparticle (FIG. 4, Table 2), but also the nanoparticles are self-assembled to form the core-shell structure nanoparticles (FIG. 4) at the size range about 200-300 nm (FIG. 5, Table 2). After 30-60 minutes, the nanoparticles were found to be stable in solution. Zeta potential analysis was used for measuring surface charge of nanoparticles. TMC precursors were highly positively charged at about 40-50 mV, and nanoparticle formation lowered the surface charge down to 15-25 mV. Nanoparticles with a high positive charge on their surface are stable in solution, which allowed sufficient antigen loading.

TABLE 2
Zeta potential of Nanoparticles synthesized at the TMC:TPP ratio of 5:1 (a) and
10:1 (b) during the reaction at 1 hour and 1 day with and without stirring.

| | | | | | Average | Average |
|---|---|---|---|---|---|---|
| | Ratio | | Reaction | Zeta | size of | assembly |
| TMC- | of | Reaction | Time | Potential | single | size of |
| TPP NPs | TMC:TPP | condition | (hours) | (mV) | NPs (nm) | NP (nm) |
| NP5-1 | 5:1 | Stirring | 1 | 16.7 ± 4.18 | 48.0 | 224.3 |
| NP5-2 | | (700 rpm) | 24 | 18.7 ± 3.85 | 58.2 | 312.8 |
| NP5-3 | | Non- | 1 | 17.1 ± 4.08 | 56.4 | 345.6 |
| NP5-4 | | stirred | 24 | 24.6 ± 3.37 | 70.3 | 453.2 |
| NP10-1 | 10:1 | Stirring | 1 | 45.2 ± 8.11 | 18.9 | n/a |
| NP10-2 | | (700 rpm) | 24 | 52.8 ± 8.35 | 22.1 | n/a |
| NP10-3 | | Non- | 1 | 43.4 ± 6.37 | n/a | n/a |
| NP10-4 | | stirred | 24 | 42.9 ± 7.03 | n/a | n/a |

Stirring process: We investigated if the stirring process would interrupt the NP formation or influence the size of NPs. Using a magnetic stirring process at 100-1500 rpm, we could produce smaller and uniform nanoparticles compared to without stirring as shown in Table 2. The size of NPs increases slightly as the reaction time increases. A stirring speed of 700 rpm was found optimal and preferred for nanoparticle production.

### Example 3. Scale Up Synthesis of Crosslinked TMC-TPP Nanoparticle

For scale-up production, the most important stage is synthesis of the precursor trimethylated chitosan (TMC). This is one of the critical steps for making the vaccine formulation herein. This step of modification of chitosan is to generate highly soluble product in order to formulate/deliver proteins as the vaccine candidate. This was a two-step synthesis to reach the goal. We started with 1 gram of chitosan Sigma-Aldrich, 448869>=75% deacetylated) and after a two-step reaction and purification, we obtained 334 mg of solid white TMC. This is a 33.4% yield, which is very similar to that initially obtained for small batch synthesis (29.2% yield). If we dissolved the TMC in PBS or DI water, we could obtain very clear and colorless solution as shown in FIG. 6. We also achieved batch-to-batch repeatability to obtain similar size range, surface charge and yield of NPs. We also successfully obtained lyophilized nanoparticles after purification. The powder could be easily reconstituted into the PBS buffer for animal administration (FIG. 6). Dynamic Light Scattering (DLS) and Zeta potential (Zetasizer, Nano ZS90, Malvern Panalytical) were used for NP size distribution and surface charge at each step of formation of TMC-TPP NP or TMC-TPP NP-protein complexes using standard protocol. In general, the sample was dispersed in aqueous solution and 1 ml of the solution was transferred into 1 mL of cuvette and inserted to Zetasizer for the measurement using Malvern application software for data processing. Following this general procedure, Zeta potential measurements were conducted to monitor the change of charges. The surface charge of pure TMC was 45.2±7.45 mV, the surface charge of crosslinked TMC NPs was decreased to 25.4±9.77 mV. The notable change of zeta potential indicates that there was an interaction between positively charged TMC and negatively charged TPP. Also, DLS measurements were conducted to measure the size for TMC-TPP NPs. The average size of TMC-TPP NPs was 280.9±128.8 nm which was consistent with what we obtained previously at small scale.

### Example 4. Microfluid System for Nanoparticle Synthesis

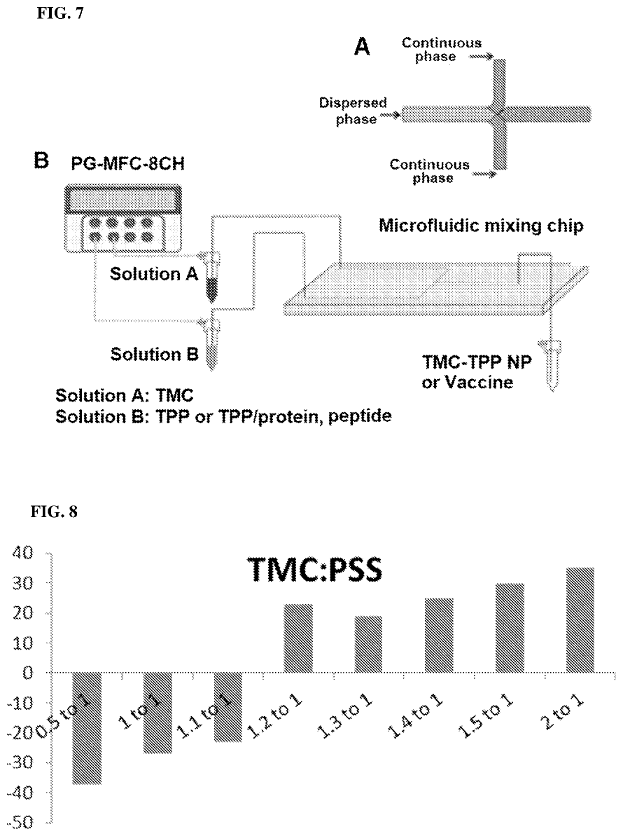

We have developed a conventional method for small scale TMC-TPP nanoparticle synthesis that was described above. However, in order to reduce the reaction time and cost while better controlling the final NP composition, size distribution, and repeatability of synthesis with potential for large scale production, we worked on transitioning production to the NanoGenerator Flex Nanoparticle Synthesis system (PG-SYN-8, PreciGenome, California). FIG. 7 illustrates the device with junction in focused-flow geometry designed for particle synthesis).

Solution A and Solution B were loaded in reservoir kits for nanoparticle production. In nanoparticle synthesis experiments, Solution A is TMC in water-based solution. Solution B is TPP in water-based solution. Different ratios of TMC and TPP were tested based on previously developed NP synthesis precursors feed ratio at different flow rate. When the positively charged chitosan and the negatively charged TPP solution are mixed in the NanoGenerator, NPs were formed via electrostatic assembly. In order to encapsulate different subunit proteins or peptides, we need to premix them with either TMC or TPP. The produced nanoparticle or vaccine solution can be collected from the outlet of the microfluidic chip after only a few seconds (˜12 secs), significantly faster than the conventional mechanical stirring method we developed that requires at least 1 hour reaction time (obtaining 1.2 mg of NPs). As a result, in one hour, we could obtain approximately 300 times the amount of NPs (˜360 mg of NPs) as we could when using the traditional synthesis methods.

TABLE 3
Parameters for TMC-TPP nanoparticle synthesis using microfluidic device.

| | TMC | TMC | TPP | TPP | | Total | | | Total | | Average |
|---|---|---|---|---|---|---|---|---|---|---|---|
| Sample | conc. | vol. | conc. | vol. | TMC/TPP | vol | TMC flow | TPP flow | flow | Size peak | size |
| No. | (mg/mL) | (mL) | (mg/mL) | (mL) | ratio | (mL) | (mL/min) | (mL/min) | (mL/min) | (nm) | (nm) |
| 1 | 2 | 1 | 2 | 0.2 | 5:1 | 1.2 | 5 | 1 | 6 | 469.5 | 381 |
| 2 | 2 | 1 | 2 | 0.2 | 5:1 | 1.2 | 2.5 | 0.5 | 3 | 193, 22, 5 | 47 |
| 3 | 1 | 1 | 2 | 0.1 | 5:1 | 1.1 | 10 | 1 | 11 | 180.2, 12.3 | 102 |
| 4 | 2 | 1 | 4 | 0.1 | 5:1 | 1.1 | 10 | 1 | 11 | 209.1, 10.7 | 300 |
| 5 | 2 | 1.5 | 4 | 0.15 | 5:1 | 1.65 | 10 | 1 | 11 | 131.5, 13.7, 2979 | 27 |
| 6 | 2 | 1 | 4 | 0.1 | 5:1 | 1.1 | 8 | 0.8 | 8.8 | 520.1 | 395 |
| 7 | 2 | 1 | 4 | 0.1 | 5:1 | 1.1 | 9 | 0.9 | 9.9 | 160.6, 14.9, 4901 | 62 |
| 8 | 2 | 1 | 4 | 0.1 | 5:1 | 1.1 | 8.5 | 0.85 | 9.35 | 511.1, 5181 | 415 |
| 9 | 2 | 1 | 4 | 0.1 | 5:1 | 1.1 | 8 | 0.8 | 8.8 | 569.4 | 467 |
| 10 | 2 | 1 | 4 | 0.1 | 5:1 | 1.1 | 7.5 | 0.75 | 8.25 | 512.5 | 413 |
| 11 | 2 | 1 | 4 | 0.1 | 5:1 | 1.1 | 7 | 0.7 | 7.7 | 577.7 | 454 |
| 12 | 2 | 1 | 4 | 0.1 | 5:1 | 1.1 | 6 | 0.6 | 6.6 | 158.7, 8, 40.0 | 164 |

First, we investigated the effect of concentration changes on the NP synthesis. We tuned the concentration between 1 mg/ml-5 mg/ml for the test, and the results indicated that a concentration of 2-2.5 mg/ml produced consistent results and NPs size distribution based on dynamic light scattering (DLS). Next, we tuned the flow rate of each precursor, in general, the procedure of NP synthesis was described as following. TMC was dissolved in ultrapure DI water at a concentration of 2 mg/ml and TPP was dissolved in DI water at a concentration of 2 mg/ml as the initial test. Later, these concentrations were tuned for the next several tests. We placed 5 ml of TMC solution (2 mg/ml) in reservoir 1, and 2 mL of TPP solution (2 mg/ml) was placed in reservoir 2. The flow rate of TMC was 5 ml/min, the flow rate of TPP was 1 ml/min, and the total flow rate was 6 mL/min. The consumed TMC solution was 1 ml, the consumed TPP solution was 0.2 ml, the ratio of TMC and TPP was 5:1, and the obtained solution was ˜1.2 ml. The reaction time was 0.2 min. After reaction, samples were evaluated by DLS for quick measurement of size distribution during the conditions. Other conditions were tested, and the average size of NPs detected from DLS are provided in Table 3.

In the experiment, we mainly tested the effect of flow rates and precursor concentrations. The results show that both parameters are very important for controlling the formation and growth of nanoparticles. When precursor concentrations were low, there was formation of many small nanoparticles. When the flow rate was lower than 6 ml/min or higher than 8 ml/min, nanoparticles have a wide size distribution or came out with multiple peaks. The best total flow rate is between 7.7 ml/min and 8.8 ml/min for concentration of precursors TPP at 4 mg/ml, but when we decreased TPP concentration to 2 mg/mL, the best flow rate would be 6 ml/min (see bolded rows in the Table 3, where we observed only a single size peak from DLS measurements).

After tuning and optimizing all the parameters, the procedure of TMC-TPP NP synthesis is as follows and can be found summarized in Table 3. First, TMC was dissolved in ultrapure DI water at a concentration of 2 mg/mL and TPP was dissolved in ultrapure DI water at a concentration of 2 mg/ml. 5 ml of TMC and 2 mL of TPP solution were then put into reservoirs 1 and 2, respectively. The flow rate of TMC was 5 mL/min, the flow rate of TPP was 1 mL/min, and the total flow rate was 6 mL/min. The consumed TMC solution was 1 mL, the consumed TPP solution was 0.2 mL for each reaction, the ratio of TMC and TPP was 5:1, and the total obtained TMC-TPP nanoparticle solution was approximately 1.2 ml. The reaction time was 0.2 min (12 seconds). After reaction, samples were evaluated by DLS for size distribution. The reaction was repeated three times in order to obtain data for repeatability using the same conditions, and the results and conditions are shown in Table 4. We obtained very consistent results from these three repeated reactions. The average of size of NP was at 206.3±18.9 nm using DLS measurement which is within our targeted size range of 200-400 nm. We prepared the powder NPs after freeze-drying overnight. We obtained 93-97% yield of NPs. We also used scanning electron microscopy (SEM) to evaluate NP size and morphology.

TABLE 4
Parameters for TMC-TPP nanoparticle synthesis (three repeats)
using microfluidic device and the results of DLS size of NPs.

| | TMC | TMC | TPP | TPP | | Total | TMC | TPP | Total | Average |
|---|---|---|---|---|---|---|---|---|---|---|
| | conc. | vol. | conc. | vol. | TMC/TPP | vol. | flow | flow | flow | size |
| Sample | (mg/mL) | (mL) | (mg/mL) | (mL) | ratio | (mL) | (mL/min) | (mL/min) | (mL/min) | (nm) |
| 1 | 2 | 1 | 2 | 0.2 | 5:1 | 1.2 | 5 | 1 | 6 | 228.1 |
| 2 | 2 | 1 | 2 | 0.2 | 5:1 | 1.2 | 5 | 1 | 6 | 197.4 |
| 3 | 2 | 1 | 2 | 0.2 | 5:1 | 1.2 | 5 | 1 | 6 | 193.4 |

### Example 5. Protective Layer (Outside Layer) Coating

In addition to the TPP crosslinked TMC nanoparticle “core” we also investigated the inclusion of a negatively charged polymer outer layer. We worked on Poly (4-styrenesulfonic acid) sodium salt (PSS), poly (allylamine hydrochloride) (PAH; MW 50K-65K) and Hyaluronate (HA, 60 K MW). We determined that PSS and HA are good candidates for LbL formulations since they show the optimized release profiles for vaccine development.

Here, as an example, the different ratio of TMC with Poly (4-styrenesulfonic acid) sodium salt (PSS, Sigma, 70 k) in formulation of NP has been evaluated. During the experiment, TMC was dissolved in DI water at a concentration of 5 mg/ml. Likewise, PSS was dissolved in DI water at a concentration of 2 mg/ml. Subsequently, an appropriate amount of TMC solution was aliquoted into a 1.5 ml centrifuge tube to yield final concentrations of a) 0.5 mg, b) 1.0 mg, c) 1.1 mg, d) 1.2 mg, e) 1.3 mg, f) 1.4 mg, g) 1.5 mg, h) 2.0 mg of TMC. Then, 500 μl of PSS solution was added to TMC, for a final concentration of 1 mg/ml and the volume was brought up to 1 ml. Solutions were vortexed for 1 hr at room temperature. The zeta potential was then measured accordingly.

The ratios tested were 1:2 to 2:1 of TMC to PSS. As shown in Table 5 and FIG. 8, the charge of NPs changed from negatively charged to positively charged when the ratio of TMC:PSS increased from 0.91:1 to 0.83:1. As a result, we will focus on ratios lower than 1:0.83 for the protein loading in the future. At the same time, we also investigated the charge changes when we tuned the ratio between TMC:TPP:PSS. As shown in Table 6, once we added PSS during the NP formulation, the surface charge of NP was significantly decreased from initial+37 mV to +5 mV which is consistent to the phenomenon that we observed previously. We found certain white aggregation formed during the NP synthesis. It indicated that adding the PSS caused a larger size of NPs formulation. Therefore, compositions with lower PSS content are preferred.

TABLE 5
Parameters of NP (TMC-PSS) and related
zeta-potential value of NPs

| Ratio of | Reaction time | TMC | TPP | PSS | zeta-potential |
|---|---|---|---|---|---|
| TMC:PSS | (min) | (mg) | (mg) | (mg) | (mV) |
| 1:2 | 60 | 0.5 | 0 | 1 | −37 |
| 1:1 | 60 | 1 | 0 | 1 | −27 |
| 1:0.91 | 60 | 1.1 | 0 | 1 | −23 |
| 1:0.83 | 60 | 1.2 | 0 | 1 | 23 |
| 1:0.77 | 60 | 1.3 | 0 | 1 | 19 |
| 1:0.71 | 60 | 1.4 | 0 | 1 | 25 |
| 1:0.67 | 60 | 1.5 | 0 | 1 | 30 |
| 1:0.5 | 60 | 2 | 0 | 1 | 35 |

TABLE 6
Parameters of NP (TMC-TPP-PSS) and
related zeta-potential value of NPs

| | | | | | zeta- |
|---|---|---|---|---|---|
| | Reaction | TMC | TPP | PSS | potential |
| (TMC:TPP:PSS) | time (min) | (mg) | (mg) | (mg) | (mV) |
| 1:0.2:0.1 | 60 | 1 | 0.2 | 0.1 | 5 |
| 1:0.2:0.25 | 60 | 1 | 0.2 | 0.25 | 4 |
| 1:0.2:0.5 | 60 | 1 | 0.2 | 0.5 | 4 |
| 1:0.2:0.75 | 60 | 1 | 0.2 | 0.75 | −23 |

### Example 6. Layer-by-Layer Antigen Encapsulation and Loading

We applied this delivery platform for protein/peptide delivery in malarial vaccine development and also tested it for RNA/DNA encapsulation/loading as the HIV-1 vaccine candidate. Below, we provided these details of using LbL NP platform in these applications.

### Example 6A. Protein Loading and Delivery

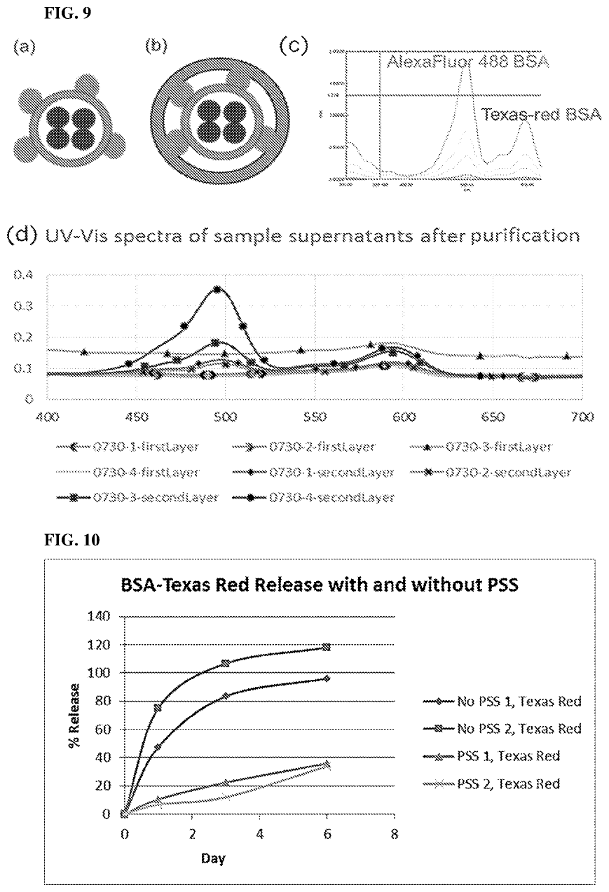

We initially used fluorescence dye labelled bovine serum albumin (BSA) as the model protein for optimizing layer-by-layer (LbL) loading and release. However, the process for loading other proteins or nucleic acids is similar. For the initial study, we chose two different dyes for the labelling of BSAs. One BSA labelled with Texas-red dye was used for encapsulation inside of the chitosan nanoparticle core, but the second layer of protein BSA with AlexaFluor 488 dye labelling was loaded on the outer layer of NP surface. Also, we had coated/added the protection layer for protein protection to compare with the one without protection layer (FIG. 9a,b). The two dye-labelled BSA proteins were evaluated using UV-Vis for quantification and identification. Results indicated that there was no interference between the absorbance of each dye once they were mixed in the solution and two individual peaks were well present in the UV-Vis spectra (FIG. 9c).

Ionic gelation is considered the most suitable method for protein loading on the LbL nanoparticles. The details for encapsulation of the BSA protein are as follows. First, the concentration of TPP solution was 2 mg/ml. Texas-red labeled BSA solution was 1 mg/ml. 2 ml of TMC solution was mixed with 1 mL and 0.5 mL of BSA solution in separated glass vials. The corresponding mass ratios of TMC to BSA are 20:1. Into each mixture solution, 50 μl of Tween™ 80 was added as non-ionic surfactant. After stirring for 10 minutes to fully mix TMC, BSA, and Tween™ 80, 2 mL of TPP solution was slowly added under constant stirring. After reaction for 1 hour, the reaction solutions were purified by gradient centrifugation with 10 μl of glycerol three times. The samples were then redispersed into DI water, and the second layer of BSA labelled by Alexa Fluor 488 was added with and without a protection layer of polystyrenesulfonate (PSS). After purification, the supernatant of the samples was measured using UV-Vis as shown in the FIG. 9 (d).

### Protective Layer of Polystyrene Sulfonate

The loading efficiency (LE) of core protein encapsulation was calculated from the UV-Vis spectra at approximately 85%. During the loading process of the second layer of protein, the first layer of protein demonstrated a burst release in solution. The LE decreased from 85% to 82% without a protective layer of PSS, while it further decreased to 74% with PSS coating. However, the loading efficiency of the second layer protein was approximately 91% without PSS and 84% with PSS. As a result, the presence of PSS was found to moderately decrease the loading amount of core and second layer proteins. This is acceptable, however, since the outer PSS layer will prevent the outer layer of protein from immediate release.

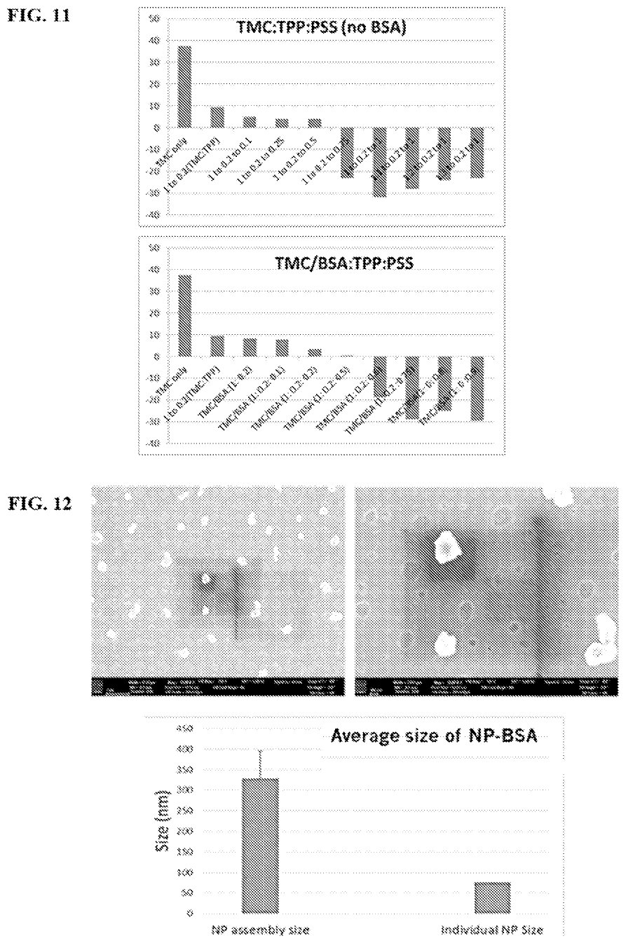

The TMC to PSS ratio was also investigated in order to optimize the formulation for protein loading as shown in FIG. 11. The ratio of TMC to PSS was tested from 1:0.75, 1:0.5, 1:0.25 and 1:0.1 (Table 6). The surface charge of NPs changed from negatively charged to positively charged when the ratio of TMC:PSS was changed from 1:0.75 to 1:0.5. Also, we found that PSS has formed a stable contact layer on the surface of protein that significantly reduced the release of the protein. In order to achieve the release profile for each protein layer, we optimized the composition of the second layer of PSS and TPP. After choosing these ratios, the BSA loaded NP complex was measured using zeta potential, and the loading efficiency and release profiles were also analyzed. The final BSA formulation loading efficiency can be as high as 91.2% or 97.5% when we applied TMC:TPP:PSS ratio of 1:0.2:0.2 or 1:0.2:0.1. Without using TPP, the protein cannot be encapsulated efficiently and tightly. PSS has a strong affinity for TMC-TPP nanoparticles during the formation of LbL NPs which allows us to use only a minimal amount to protect the outer layer of protein from immediate release.

As a result, if we applied PSS as the protective layer, the amount of PSS needs to be limited to less than 0.05 mg per mg of TMC. Also, during the release test, the burst release for these samples (two repeats for each sample) were approximately 30-40%. The releases of formulation with and without protection from PSS were measured as shown in FIG. 10, the encapsulated protein was released significantly faster. Also, we measured the size changes after loading of protein. The protein-loaded NPs were approximately 320 nm in size, as shown in the SEM images FIG. 12. This makes them appropriate candidates for intramuscular injection. The NP without self-assembly and protein loading is approximately 75 nm size, as shown in FIG. 12. The NP zeta potential was reduced from 48.4 mV to 3.5 mV, 7.8 mV and 7.3 mV once the PSS was added at 0.2 mg, 0.1 mg and 0.05 mg, respectively. This slightly positively charge NPs could easily be taken up by cells, but lower the cytotoxicity, and increased the blood compatibility, thus delivery of antigen more efficiently. During the formulation of TMC-BSA-TPP NP, PSS was added to form a protective layer. We expected this layer could prolong the release of BSA protein from NP to achieve the monthly release profile layer by layer. Then zeta potential was measured for all of these conditions. It was determined that the ratio of TMC:TPP:PSS should be kept below 1:0.2:0.5 for the BSA loading. The BSA loaded NP complex prepared under these conditions were characterized using zeta potential measurements, and determination of loading efficiency and release profiles. The final BSA formulation loading efficiency was 91.2-97.5%. However, the burst release for these samples (two repeats for each sample) are about 30-40%. Also, the release profiles of formulations with and without protection from PSS were measured as shown in FIG. 10, which shows that the protective PSS layer is necessary and required to keep the proteins in sustained releases.

### Protective Layer of Hyaluronate

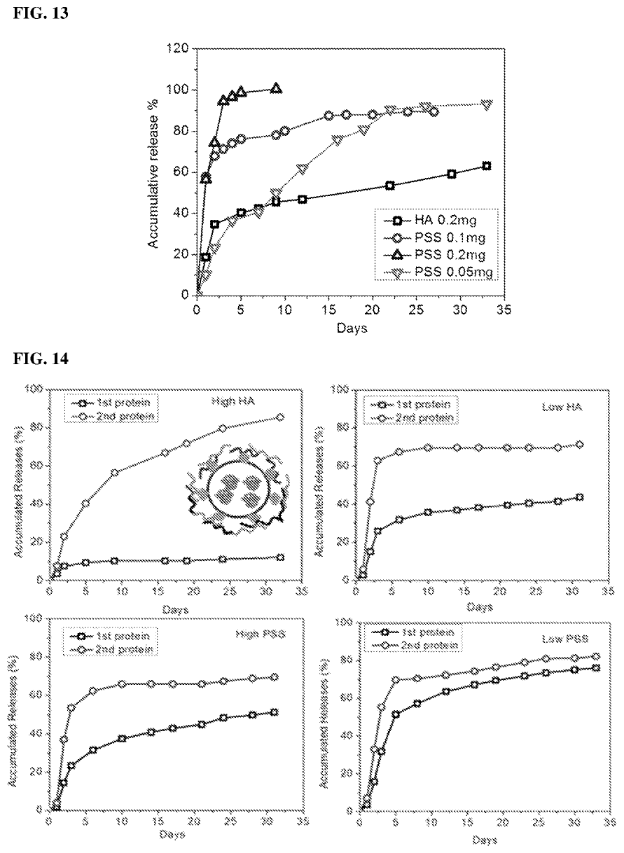

Another negatively charged polymer, Hyaluronate (HA, 60 K MW), which has lower affinity to the TMC, was also selected for comparison with PSS. Two types of HA with different molecular weight (600 K and 60 K) have been tested. The final loading efficiency for BSA protein formulations was as high as 83-96% when we applied the TMC:TPP:PSS or TMC:TPP:HA ratio of 1:0.2:0.2-1:0.2:0.05. The test for the formulation release was monitored for more than a month, with results shown in FIG. 13. The burst release for these samples (two repeats for each sample) are 30-40% for the higher PSS contents, but when we decreased the amount of PSS to 0.05 mg or for HA samples, the burst release was decreased to 10% and 24%, respectively. As shown in FIG. 13, the encapsulated protein was released significantly faster without the PSS layer, and here, we found the release rate was decreased with the decreases of PSS as the protective layer but limited between 0.05-0.1 mg per 1 mg TMC. In conclusion, the protective layer is necessary and required for long-term release profiles. The HA coating has demonstrated a very long release profile; even after a month, only about 60% of protein was released. HA could be used for long-term release formulations.

TABLE 7
Parameters of NP composition for the vaccine candidates

| | | | | | | | BSA | | Zeta |
|---|---|---|---|---|---|---|---|---|---|
| | TMC | TPP | PSS | HA | BSA | LE | (green, | LE | potential |
| Sample | (mg) | (mg) | (mg) | (mg) | (red, mg) | (%) | mg) | (%) | (mV) |
| High PSS | 1 | 0.2 | 0.05 | | 0.25 | 98.5 | 0.25 | 93.4 | 5.0 |
| Low PSS | 1 | 0.2 | 0.01 | | 0.25 | 74.2 | 0.25 | 64.3 | 12.1 |
| High HA | 1 | 0.2 | | 0.05 | 0.25 | 97.0 | 0.25 | 97.3 | 3.0 |
| Low HA | 1 | 0.2 | | 0.01 | 0.25 | 70.2 | 0.25 | 54.6 | 10.5 |

In all, through many tests, for both the first and second layer the encapsulation efficiency for BSA protein was as high as 98.47% and 93.40% (Table 7). Over the first 4 days, both protein layers had similar release profiles. However, after a week of release, the outer layer of protein (“2nd Protein”) demonstrated an increased release rate as compared to the inner core layer of protein (“1st protein”). The result of this release testing is shown in FIG. 14 to compare the performance of these formulations. It was clear that the lower the amount of protective polymer used (0.01 mg), the faster the release rate. The inner layer (first protein) release reached 42% of total after 30 days for the lower protective layer HA samples, as compared to only 15% of release when we used higher amount of protective coating of HA. However, the second protein was almost entirely released for higher HA samples. For low PSS coating, the release increased to higher than 80% at 30 days. However, the higher PSS coating, the first protein was released less amount (40%) compared to low PSS coating one with almost 80% releases. As a result, the high HA coating was set as one of our vaccine candidates as shown in the FIG. 14. These results indicated that we could control the protein release profile by tuning the protective layer of coating.

### Example 7. Synthesis of TMC/FTIC-Peptide-TPP NPs Using Microfluidic Device

As discussed in the previous section, the microfluidic setup was used to reduce the reaction time and better control the final vaccine candidate composition, size distribution, and repeatability of synthesis with potential for large scale production. This system can be applied for peptide or protein encapsulation. As an example, we have encapsulated the peptide Fluorescein Isothiocyanate dye (FTIC) labelled VVFLHVTYV which targets the SARS-CoV-2 CD8+ T-Cell responses.

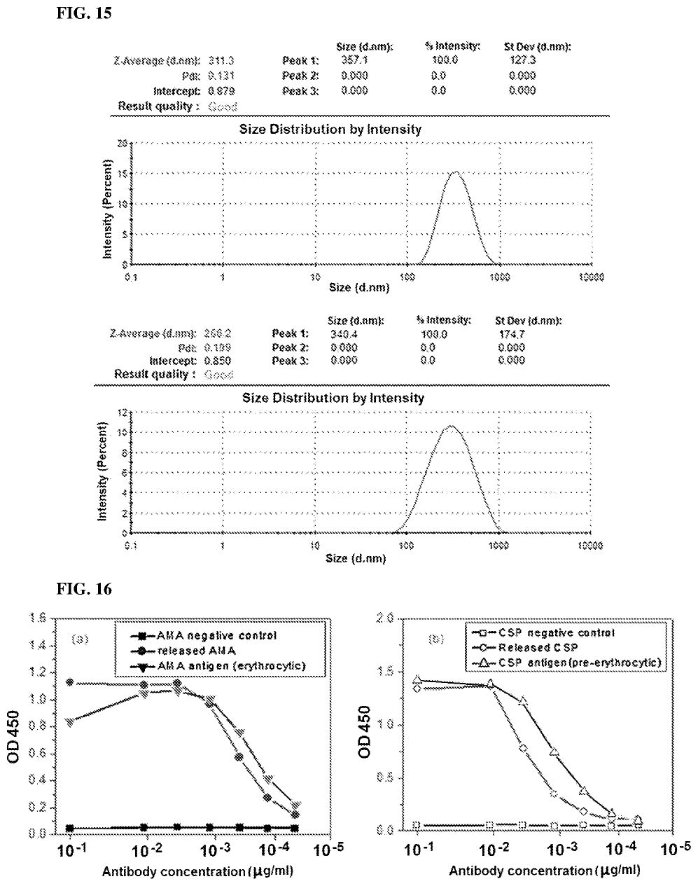

The method was very similar to the one described for NP synthesis. TMC precursor was dissolved in ultrapure DI water at a concentration of 5 mg/ml, which was diluted to different concentrations when needed. Peptide was dissolved in DMSO at a concentration of 40 mg/ml. Peptide and TMC solution were premixed at 1:5 ratio and then 5 ml of TMC-peptide solution was placed in reservoir 1 of microfluidic device and 2 ml of TPP solution (1.5 mg/ml) was placed in reservoir 2. The flow rate of TMC-peptide was 5 ml/min, the flow rate of TPP was 1 ml/min, and the total flow rate was 6 ml/min. The consumed TMC-peptide solution was 1 ml, the consumed TPP solution was 0.2 ml, the ratio of TMC and TPP was 5:1, and the obtained solution was ˜1.2 ml. The reaction time was 0.2 min (12 seconds). After reaction, samples were evaluated by DLS measurements. The reaction was repeated two times and the DLS measurements were performed to identify the nanoparticle sizes. The size of TMC-peptide-TPP nanoparticle sizes were 311.3±127.3 nm and 266.2±174.7 nm in these two reactions (FIG. 15). The size of TMC-TPP-peptide nanoparticles were larger than TMC-TPP nanoparticle (Table 4) which indicates the successful encapsulation of peptide.

### Example 8. Evaluate the Antigenicity and Integrity of Antigen Loaded Chitosan NPs Complexes and Antigenicity and Integrity Studies

To develop a more effective malaria vaccine with protective immune response and delivery of multiple life cycle stage antigens, we here adapt our LbL nanoparticle that enables the delivery and LBL release of multiple malaria antigens in a controllable manner. We have successfully constructed the LbL nanoparticle that efficiently loads d of different stage of antigens. It encapsulates blood stage of antigen Plasmodium Falciparum malaria parasite apical membrane antigen PfAMA-1 and/or merozoite surface protein (PfMSP-1) inside the core and also absorbs and stabilizes the pre-erythrocytic stage of antigen PfCSP (full length) on the out layer as the LbL nanoparticle formulations. To confirm that the antigenicity of each protein will not be altered following entrapment or loading on the chitosan surface using the method of production herein, an enzyme-linked immunosorbent assay (ELISA) was used to evaluate the effect of the preparation process influence on released protein functions. After each step of loading of different antigens on trimethylated chitosan nanoparticle, the antigens were released. We collected released proteins to evaluate properties. ELISA results demonstrated that both free and entrapped protein after release from NPs possessed similar responses to their antibodies as shown in the FIG. 16.

### Example 9. Nucleic Acids Loaded Chitosan NPs

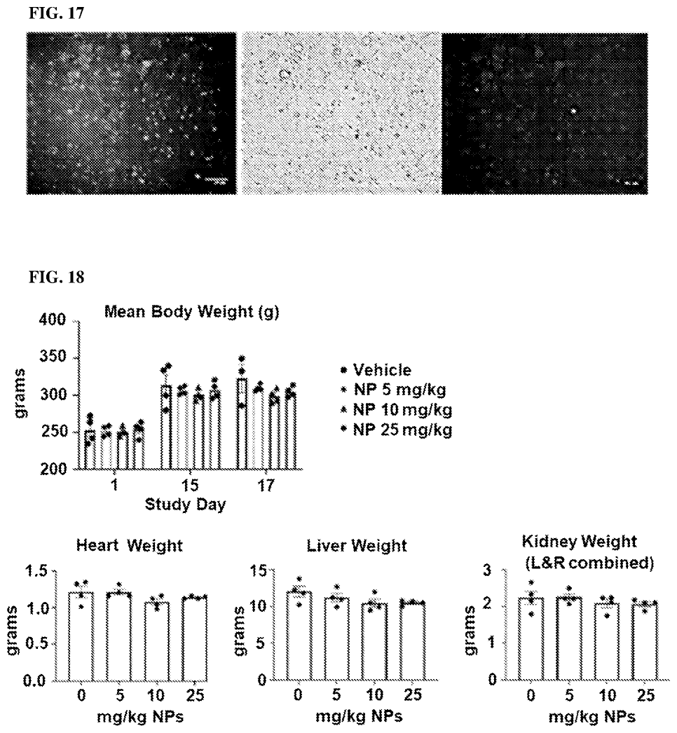

Our chitosan-based LbL platform can be used to carry genetic materials including plasmid DNA (pDNA), oligonucleotides mRNA and siRNA. Unlike other cationic polymers, chitosan has several advantages such as low toxicity, excellent biocompatibility as well as a high positive charge. Similarly, chitosan can form complexes with negatively charged genes easily due to its abundant amine groups. However, clinical translation of chitosan-based gene delivery carriers is still unsatisfactory due to several challenges including poor water solubility at physiological pH and poor targeting capability. However, we modified the chitosan to produce TMC, which was soluble in aqueous solution over a wide range of pH. For example, we performed an assay to determine mRNA transfection efficiency encapsulated by LbL TMC-TPP NPs. In this study. the transfection of a reporter EGFP and mCherry mRNA which were synthesized by TriLink Biotechnologies to encode for EGFP protein and mCherry protein. EGFP and mCherry were selected as model mRNAs to provide insights about transfection that can be applied to other mRNAs. We tested the transfection of two mRNAs with NPs using APC cells (Dendritic cells). We demonstrated excellent transfection efficiency for both mRNA models (FIG. 17) using TMC-TPP NP as potential gene delivery vector and adjuvant.

### Example 10. Animal Safety Studies

We evaluated the tolerability of a trimethylated chitosan nanoparticle (TMC/TPP-NPs) administered intramuscularly to male Sprague-Dawley rats twice over 14 days. In total, sixteen (16) male Sprague-Dawley rats were assigned to 4 groups (vehicle control or 3 dose levels of nanoparticle [n=4/group]) as shown in Table 8 below.

TABLE 8
Study schedule and dose ranges for IM administration of rat.

| | | | | TMC/TPP- |
|---|---|---|---|---|
| Group | Sex | n | Animal No. | NPs (mg/kg) |
| 1 (control) | Male | 4 | 1001-1004 | 0 (vehicle) |
| 2 (low) | Male | 4 | 2001-2004 | 5 |
| 3 (moderate) | Male | 4 | 3001-3004 | 10 |
| 4 (high) | Male | 4 | 4001-4004 | 25 |

Clinical observations were recorded up to once daily and body weights were assessed prior to dosing and at least twice weekly thereafter. The tissues/organs were also collected and weighed from all animals: heart, liver, kidney, and muscle tissues at the site of administration. Tissues/organs were processed using standard H&E staining. Microscopic evaluations of tissues/organs were conducted by a qualified Veterinary Pathologist. There were no drug (LbL NPs)-related clinical findings. Several animals exhibited bruising or scabbing of the tail as a result of tail vein blood collections. There were also no statistically significant differences in mean body weights between groups on Day 1, 15, and 17 (FIG. 18). In addition, there were no differences in heart, liver, or kidney weight between groups. LbL NPs at higher than 5 mg/kg caused muscle pathology at the site of injection when administered on Days 1 and 15 intramuscularly using a dose volume of 0.5 ml/kg to male Sprague-Dawley rats.

### Example 11. Mouse Immunogenicity Studies of Chitosan Loaded Multiple Stage Antigen Releases

The goal of using LbL TMC-TPP nanoparticle was to determine whether the LbL vaccine formulations will enhance the immunogenicity of malaria antigens and help elicit the specific immune responses in vivo. Mouse sera were collected for serology analysis of the antibody titers of pre-erythrocytic protective antigen CSP, blood stage protective antigen MSP-1 and AMA-1 for each formulation and the numbers of IFN-γ-secreting T cells in spleens of mice immunized with antigens by intramuscular injection were measured by IFN-γ enzyme linked immunospot (ELISPOT) assay.

We formulated vaccine candidates by loading CSP and AMA-1, MSP-1 in the TMC-TPP LbL structure. Three formulations were delivered for the animal studies for two doses or three doses by intramuscular injections. They were NP-CSP (TMC-TPP encapsulated CSP); NP-AMA-1/MSP-1 (TMC-TPP encapsulated MSP-1 inside of core, and AMA-1 in the outside layer); NP-CSP/AMA-1/MSP-1 (TMC-TPP encapsulated MSP-1 with second layer of AMA-1 and the outside layer is CSP). The goal was to release a shell of antigen CSP first and the core antigens after 3-4 weeks. Also, another two adjuvants ISA 720 (purchased from Seppic Inc.), a natural metabolizable nonmineral oil and a highly refined emulsifier of mannite monooleate family and 7DW8-5, a recently identified novel analog of α-galactosylceramide (α-GalCer), that enhances the level of malaria-specific protective immunity, were incorporated with these NP formulations for the comparison tests.

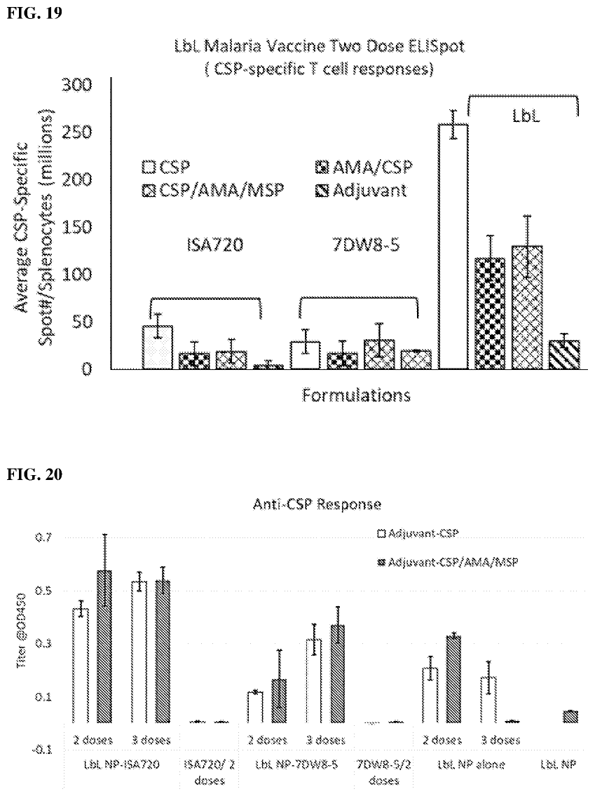

From the cellular responses (FIG. 19) and ELISA results shown in FIG. 20, it was demonstrated that the NP has shown an adjuvant effect. Especially in the group with adjuvant ISA 720, it produced the highest humoral responses for both two and three dose sera samples. In the two-dose case, this should be sufficient to induce high enough response compared to three doses no matter which antigen was evaluated. In the NP group without extra adjuvant, we found the three-protein loaded formulation with two doses injection has shown the highest humoral responses, however, by NP alone as the negative control, ELISA results of sera of mice seemed to induce a low titer of antibody against PfCSP and potentially also against AMA-1.

Results from the ELISPOT CD4 T cell response study demonstrated that NP group formulations showed much greater responses in CSP-specific CD4 T cells as shown in both 2-dose and 3-dose results than the other two adjuvant groups (ISA72 and 7DW8-5). In the 3 dose results, these responses continued to increase for the NP-CSP formulations. The other two adjuvant groups which served as controls both demonstrated a very low response. The NP vaccine candidate group alone induced the highest PfCSP specific T-cell response. This may indicate that chitosan nanoparticles have a sugar like structure similar to the PfCSP sugar structure which causes the cellular and humoral responses. The NP vector is such a potent immunogen that it may induce antibodies that have reactivity to CSP or even to AMA-1. Compared between two doses and three doses, we believed that 3 doses of immunization with NP vaccine may cause immunosuppression. So far, we have concluded that the NP vector is an extremely potent vaccine vector. Two doses of immunization with a longer interval between them, likely 4 weeks, should induce the highest humoral response against CSP.

### Example 12. Perform Protective Efficacy Studies by Parasite Challenge in Mouse Model

We evaluated the efficacy of the optimal NPs-antigen to protect against sporozoite challenge in a mouse model and using an in vitro growth inhibition activity (GIA) assay. Intramuscular injection was used as the administration route and malaria proteins were formulated using the trimethylated chitosan nanoparticles herein and other two adjuvants for comparison. The PfCSP/Py sporozoite which was obtained from Sanaria was used for challenging vaccinated BALB/c mice by intravenous injection.

TABLE 9
Protection of mice immunized with vaccine candidates against
transgenic PfCSP/Py sporozoites administrated intravenously.

| | Protected/ | |
|---|---|---|
| Vaccine formulations | Challenged | p-Value |
| Group 1: Naive | 0/6 | |
| Group 2: CSP + ISA720 | 6/6 | 0.061a |
| Group 3: CSP/AMA1/MSP1 + ISA720 | 2/6 | 1a; 0.24b |
| Group 4: ISA720 only | 2/6 | |
| Group 5: CSP + 7DW8-5 | 5/6 | 0.008a*; 0.015b* |
| Group 6: CSP/AMA1/MSP1 + 7DW8-5 | 4/6 | 0.24a; 0.061b |
| Group 7: 7DW8-5 only | 1/6 | |
| Group 8: CSP + LbL NPss | 5/6 | 0.008a*; 0.015b* |
| Group 9: CSP/AMA1/MSP1 + LbL NPs | 4/6 | 0.24a; 0.061b |
| Group 10: LbL NPs only | 1/6 | |

aThe p value between vaccine candidate group to each adjuvant group
bThe p value between vaccine candidate group to naïve group
*p < 0.05 significant

Mice immunized with CSP alone (groups 2, 5, 8, Table 9) were 83-100% protected compared to naïve mouse group, indicating that these single CSP vaccines displayed good efficacy. Immunization with CSP/AMA1/MSP1 with 7DW8-5 (group 6) and NP-CSP/AMA1/MSP1 (group 9) also induced moderate protection (66.7%) compared to the naïve mouse group, resulting in protecting four out of six mice from infection with malaria. When we compared these vaccinated groups with internal control groups (adjuvant alone groups 7 and 10). CSP+7DW8-5 (group 5), and NP-CSP (group 8) are still able to show a statistically significant efficacy (p<0.05, Fisher's test). However, immunization with CSP/AMA1/MSP1 with ISA720 (group 3) was able to protect only two out of six mice (33.3%), which was identical to that seen in mice immunized with ISA720 alone (group 4). The reason why CSP alone seems more potent is because when you combine more than one protein, the presentation of one antigen could be slightly diminished due to the competition at the level of antigen-presentation (multiple proteins will compete for MHC class I and class II-mediated presentation). In our current study, we challenged with P. yoelii parasites that express only PfCSP, and, therefore, we saw the protective immune response targeted against PfCSP only. However, if we establish PfCSP/PfAMA-1/PfMSP-1 triple transgenic parasites and challenge them, the NP expressing the three proteins may exert a better efficacy compared to a single protein-expressing NP vaccine. Although the protective immunity induced by PfCSP (one antigen) may be weaker, a combined protective immunity induced by all 3 proteins may be more potent due to additive or synergistic effect. Group 4 had two uninfected mice. It is possible that ISA720 may elicit innate immune response that was potent enough to mediate non-specific anti-malarial effect. Also, it is rare to see protection lasting for more than 4 weeks after administration of a booster. In other words, there have been no other malaria vaccines found that can sustain this sterile protection for more than 2 weeks.

We therefore believe our LbL NP vaccine candidates are not only potent but also very long-lasting. The adjuvants can elicit innate immune response that was potent to mediate non-specific anti-malarial effect.

### REFERENCES

1. A. L. Z. Lee, C. Yang, S. Gao, Y. Wang, J. L. Hedrick, Y. Y. Yang. 2020. “Biodegradable cationic polycarbonates as vaccine adjuvants.” ACS Appl. Mater. Interfaces 12: 52285-52297.
2. Di Pasquale A, Preiss S, Tavares Da Silva F, Garçon N. 2015. “Vaccine Adjuvants: from 1920 to 2015 and Beyond.” Vaccines (Basel) 16 (3): 320-342.
3. Genç, R, N Yakubogullari, A Nalbantsoy, F Çöven, and Bedir E. 2020. “Adjuvant potency of astragaloside vii embedded cholesterol nanoparticles for H3N2 influenza vaccine.” Turk. J. Biol. 304-314.
4. Jabbal-Gill, I. 2010. “Nasal vaccine innovation.” J Drug Target. 18 (10): 771-786.
5. Kritchenkov A. S., Andranovitš S., Skorik Y. A. Russ. 2017. “Chitosan and its derivatives: Vectors in gene therapy.” Chem. Rev. 86: 231-239.
6. Lai, P., Dacar, W., Lobenberg, R., & Prenner, E. 2014. “Overview of the preparation of organic polymeric nanoparticles for drug delivery based on gelatine, chitosan, poly(d,l-lactide-co-glycolic acid) and polyalkylcyanoacrylate.” Colloids Surf B Biointerfaces 154-163.
7. Lee, Ashlynn L Z, C Yang, S Gao, Y Wang, James L Hedrick, and Y Y Yang. 2020. “Biodegradable cationic polycarbonates as vaccine adjuvants.” ACS Appl. Mater. Interfaces 52285-52297.
8. Niels Hagenaars, Rolf J. Verheul, Imke Mooren, Pascal H. J. et al. 2009. “Relationship between structure and adjuvanticity of N,N,N-trimethyl chitosan (TMC) structural variants in a nasal influenza vaccine.” Journal of Controlled Release 126-133.
9. S. Bashiri, P. Koirala, I. Toth, M. Skwarczynski., 2020. “Carbohydrate immune adjuvants in subunit vaccines.” Pharmaceutics 12: 1-33.
10. Shakya, A. K., and K. S. Nandakumar. 2012. “Polymers as Immunological Adjuvants: An Update on Recent Developments.” J. Biosci, Biotechnol. 1 (3): 199-210.
11. Tafaghodi, M, V Saluja, GF Kersten, H Kraan, B Slütter, and J-P. Amorij. 2012. “Hepatitis B surface antigen nanoparticles coated with chitosan and trimethyl chitosan: Impact of formulation on physicochemical and immunological characteristics.” Vaccine 5341-5348.
12. Watts, P, and A Smith. 2014. “ChiSys® as a Chitosan-Based Delivery Platform for Nasal Vaccination.” In Mucosal Delivery of Biopharmaceuticals, 499-516. Springer.
13. Xu, Y., Du, Y. 2013. “Preparation and modification of N-(2-hydroxyl) propyl-3-trimethyl ammonium chitosan chloride nanoparticle as a protein carrier.” Biomaterials 5015-5022.
14. Yan, X, M Zhou, S Yu, Z Jin, and K. Zhao. 2020. “An overview of biodegradable nanomaterials and applications in vaccines,” Vaccine. 38: 1096-1104.

The Summary and Abstract sections may set forth one or more but not all exemplary embodiments of the present invention as contemplated by the inventor(s), and thus, are not intended to limit the present invention and the appended claims in any way.

The present invention has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

With respect to aspects of the invention described as a genus, all individual species are individually considered separate aspects of the invention. If aspects of the invention are described as “comprising” a feature, embodiments also are contemplated “consisting of” or “consisting essentially of” the feature.

The foregoing description of the specific embodiments will so fully reveal the general nature of the invention that others can, by applying knowledge within the ordinary skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the ordinarily skilled artisan in light of the teachings and guidance.

The breadth and scope of the present invention should not be limited by any of the above-described exemplary embodiments.

All of the various aspects, embodiments, and options described herein can be combined in any and all variations.

All publications, patents, and patent applications mentioned in this specification are herein incorporated by reference to the same extent as if each individual publication, patent, or patent application was specifically and individually indicated to be incorporated by reference. To the extent that any meaning or definition of a term in this document conflicts with any meaning or definition of the same term in a document incorporated by reference, the meaning or definition assigned to that term in this document shall govern.

What is claimed is:

1. A pharmaceutical composition comprising nanoparticles having a core-shell structure, wherein the nanoparticles comprise a crosslinked polymer comprising a cationic chitosan and an anionic cross-linker, wherein the nanoparticles have an average particle size of about 40 nm to about 1 μm as determined by Dynamic Light Scattering.

2. The pharmaceutical composition of claim 1, wherein the cationic chitosan comprises quaternized ammonium cations.

3. The pharmaceutical composition of claim 1 or 2, wherein the cationic chitosan is water soluble at a neutral pH, preferably, the cationic chitosan has an aqueous solubility at least 10 mg/ml at pH 5-8.

4. The pharmaceutical composition of any of claims 1-3, wherein the cationic chitosan is N-trimethylated chitosan, with a degree of quaternization of between about 20% to about 60%, as determined by 1H-NMR.

5. The pharmaceutical composition of any of claims 1-4, wherein the cationic chitosan is prepared by treating a chitosan with a methylating agent (e.g., MeI), wherein the chitosan is characterized as having a degree of deacetylation of 75-85% and an average viscosity molecular weight (Mv) of about 50,000-190,000 Daltons.

6. The pharmaceutical composition of any of claims 1-5, wherein the anionic cross-linker is tripolyphosphate.

7. The pharmaceutical composition of any of claims 1-6, wherein the nanoparticles comprise N-trimethylated chitosan and tripolyphosphate, with a weight ratio of N-trimethylated chitosan to tripolyphosphate ranging from about 2:1 to about 20:1, preferably, about 5:1 to about 10:1, more preferably, about 5:1 to about 7:1.

8. The pharmaceutical composition of any of claims 1-7, wherein the nanoparticles further comprise a surfactant, such as a non-ionic surfactant, e.g., Tween™ 80.

9. The pharmaceutical composition of any of claims 1-8, wherein the nanoparticles have an average particle size of about 40 nm to about 500 nm, or 150 nm to about 500 nm, preferably, about 200 nm to about 400 nm, as determined by Dynamic Light Scattering.

10. The pharmaceutical composition of any of claims 1-9, further comprising at least one active agent, which is encapsulated within the nanoparticles and/or adsorbed on the surface of the nanoparticles.

11. The pharmaceutical composition of claim 10, wherein the at least one active agent is a small molecule drug, a protein, a nucleic acid, or a vaccine, or an adjuvant, preferably, the active agent is negatively charged (PI<7) at pH 7 or higher, or a hydrophobic molecule, such as a small molecule drug having a Log P of greater than 1, e.g., 1-5.

12. The pharmaceutical composition of any of claims 1-11, wherein the nanoparticles further comprise a coating layer.

13. The pharmaceutical composition of claim 12, wherein the coating layer comprises a negatively charged biocompatible polymer.

14. The pharmaceutical composition of claim 13, wherein the coating layer comprises hyaluronic acid salt (e.g., sodium hyaluronate) or polystyrene sulfonate (e.g., sodium polystyrene sulfonate).

15. The pharmaceutical composition of any of claims 1-14, wherein the nanoparticles have a zeta potential ranging from about −40 mV to about 50 mV.

16. The pharmaceutical composition of any of claims 12-15, wherein the coating layer is present in an amount such that the weight ratio of the cationic chitosan (e.g., N-trimethylated chitosan) to the coating layer is in the range of about 1:1 to about 200:1, such as about 5:1 to about 20:1.

17. The pharmaceutical composition of any of claims 12-15, wherein (1) the nanoparticles comprise N-trimethylated chitosan and tripolyphosphate, with a weight ratio of N-trimethylated chitosan to tripolyphosphate ranging from about 2:1 to about 20:1, preferably, about 5:1 to about 10:1, more preferably, about 5:1 to about 7:1; and (2) the coating layer comprises polystyrene sulfonate (e.g., sodium polystyrene sulfonate), and the weight ratio of N-trimethylated chitosan to polystyrene sulfonate ranges from about 1:1 to about 200:1, preferably about 5:1 to about 50:1, such as about 10:1 or 20:1.

18. The pharmaceutical composition of any of claims 12-15, wherein (1) the nanoparticles comprise N-trimethylated chitosan and tripolyphosphate, with a weight ratio of N-trimethylated chitosan to tripolyphosphate ranging from about 2:1 to about 20:1, preferably, about 5:1 to about 10:1, more preferably, about 5:1 to about 7:1; and (2) the coating layer comprises hyaluronic acid salt (e.g., sodium hyaluronate), and the weight ratio of N-trimethylated chitosan to hyaluronic acid salt ranges from about 1:1 to about 200:1, preferably about 5:1 to about 50:1, such as about 10:1 or 20:1.

19. A modified release formulation comprising:
(1) drug-loaded nanoparticles having a core-shell structure, wherein the drug-loaded nanoparticles comprise a crosslinked polymer comprising a cationic chitosan and an anionic cross-linker, a first active agent, and a second active agent; and
(2) a layer coating the drug-loaded nanoparticles;
wherein the first active agent is encapsulated within the drug-loaded nanoparticles and the second active agent is adsorbed on the surface of the drug-loaded nanoparticles, wherein the first and second active agents can be the same or different active agents, and
wherein the drug-loaded nanoparticles have an average particle size of about 40 nm to about 1 μm as determined by Dynamic Light Scattering.

20. The modified release formulation of claim 19, wherein the cationic chitosan comprises quaternized ammonium cations.

21. The modified release formulation of claim 19 or 20, wherein the cationic chitosan is water soluble at a neutral pH, preferably, the cationic chitosan has an aqueous solubility of at least 10 mg/ml at pH 5-8.

22. The modified release formulation of any of claims 19-21, wherein the cationic chitosan is N-trimethylated chitosan, with a degree of quaternization of between about 20% to about 60%, as determined by 1H-NMR.

23. The modified release formulation of any of claims 19-22, wherein the cationic chitosan is prepared by treating a chitosan with a methylating agent (e.g., MeI), wherein the chitosan is characterized as having a degree of deacetylation of 75-85% and an average viscosity molecular weight (Mv) of about 50,000-190,000 Daltons.

24. The modified release formulation of any of claims 19-23, wherein the anionic cross-linker is tripolyphosphate.

25. The modified release formulation of any of claims 19-24, wherein the drug-loaded nanoparticles comprise N-trimethylated chitosan and tripolyphosphate, with a weight ratio of N-trimethylated chitosan to tripolyphosphate ranging from about 2:1 to about 20:1, preferably, about 5:1 to about 10:1, more preferably, about 5:1 to about 7:1.

26. The modified release formulation of any of claims 19-25, wherein the first and second active agents are independently a small molecule drug, a protein, a peptide, a nucleic acid, or a vaccine or a therapeutic agent, preferably, the first and/or second active agents are negatively charged (PI<7) at pH 7 or higher, or the first and/or second active agents are hydrophobic molecules such as those small molecule drugs having a Log P of at least 1, e.g., 1-5.

27. The modified release formulation of any of claims 19-26, wherein the drug-loaded nanoparticles comprise the first and second active agents in a total amount of about 10-100% by weight of the cationic chitosan.

28. The modified release formulation of any of claims 19-27, wherein the coating layer comprises a negatively charged biocompatible polymer.

29. The modified release formulation of any of claims 19-27, wherein the coating layer comprises hyaluronic acid salt (e.g., sodium hyaluronate) or polystyrene sulfonate (e.g., sodium polystyrene sulfonate).

30. The modified release formulation of any of claims 19-27, wherein the coating layer is present in an amount such that the weight ratio of the cationic chitosan (e.g., N-trimethylated chitosan) to the coating layer is in the range of about 1:1 to about 200:1, such as about 5:1 to about 20:1.

31. The modified release formulation of any of claims 19-27, wherein (1) the drug-loaded nanoparticles comprise N-trimethylated chitosan and tripolyphosphate, with a weight ratio of N-trimethylated chitosan to tripolyphosphate ranging from about 2:1 to about 20:1, preferably, about 5:1 to about 10:1, more preferably, about 5:1 to about 7:1; and (2) the coating layer comprises polystyrene sulfonate (e.g., sodium polystyrene sulfonate), and the weight ratio of N-trimethylated chitosan to polystyrene sulfonate ranges from about 1:1 to about 200:1, preferably about 5:1 to about 50:1, such as about 10:1 or 20:1.

32. The modified release formulation of any of claims 19-27, wherein (1) the drug-loaded nanoparticles comprise N-trimethylated chitosan and tripolyphosphate, with a weight ratio of N-trimethylated chitosan to tripolyphosphate ranging from about 2:1 to about 20:1, preferably, about 5:1 to about 10:1, more preferably, about 5:1 to about 7:1; and (2) the coating layer comprises hyaluronic acid salt (e.g., sodium hyaluronate), and the weight ratio of N-trimethylated chitosan to hyaluronic acid salt ranges from about 1:1 to about 200:1, preferably about 5:1 to about 50:1, such as about 10:1 or 20:1.

33. The modified release formulation of any of claims 19-32, wherein the drug-loaded nanoparticles further comprise a surfactant, such as a non-ionic surfactant, e.g., Tween™ 80.

34. The modified release formulation of any of claims 19-33, wherein the drug-loaded nanoparticles have an average particle size of about 40 nm to about 600 nm, or about 150 nm to about 500 nm, preferably, about 200 nm to about 400 nm, as determined by Dynamic Light Scattering.

35. The modified release formulation of any of claims 19-34, wherein the coated drug-loaded nanoparticles have a zeta potential ranging from about −40 mV to about 50 mV.

36. The modified release formulation of any of claims 19-35, wherein about 10-50% of the second active agent is released over a burst release period of about 24 hours to about 4 days.

37. The modified release formulation of any of claims 19-36, wherein about 50-90% of the first active agent is released over a period of about 30 days.

38. The modified release formulation of any of claims 19-37, in the form of a solution, gel, dispersion, or suspension.

39. The modified release formulation of any of claims 19-37, which is a solid or liquid dosage form, such as dry powder, tablets, capsules, solution, gel, dispersion or suspension, etc.

40. A method of preparing the nanoparticles according to any of claims 1-18, the method comprising mixing the cationic chitosan and the anionic cross-linker in an aqueous solution.

41. The method of claim 40, wherein the mixing comprises stirring the cationic chitosan and the anionic cross-linker in the aqueous solution at a speed of about 100-1500 rpm for a period of about 15 minutes to 24 hours.

42. The method of claim 40, wherein the mixing comprises mixing a solution of the cationic chitosan and a solution of the anionic cross-linker in a microfluid system.

43. The nanoparticles prepared by any of the methods according to claims 40-42.

44. A method of preparing the modified release formulation according to any of claims 19-39, the method comprising (1) mixing the cationic chitosan, the anionic cross-linker, and the first active agent to form core-shelled nanoparticles encapsulating the first active agent; (2) mixing the core-shelled nanoparticles obtained in (1) with the second active agent to form the drug-loaded nanoparticles with the second active agent adsorbed on the surface of the drug-loaded nanoparticles; and (3) coating the drug-loaded nanoparticles.

45. The modified release formulation obtained by the method according to claim 44.

46. A method of stabilizing an active agent for storage comprising (1) mixing a cationic chitosan, an anionic cross-linker, and the active agent to form core-shelled nanoparticles encapsulating the active agent; and optionally (2) coating the core-shelled nanoparticles obtained in (1).

47. The method of claim 46, wherein the active agent is a negatively charged agent, such as a negatively charged protein, antigen, drug molecules, antibodies, etc. or the active agent is a hydrophobic molecule, such as a small molecule drug having a Log P of greater than 1, e.g., 1-5.

48. A method of delivering one or more active agents to a subject in need thereof, the method comprising administering to the subject the modified release formulation according to any of claims 19-39 and 45.

49. The method of claim 48, wherein the administering comprises intramuscular or subcutaneous injection of the modified release formulation.

50. The method of claim 48, wherein the administration of the modified release formulation is through transdermal or transmucosal route, such as oral or intranasal.

51. A method of delivering a vaccine to a subject in need thereof, the method comprising administering the subject the pharmaceutical composition according to any of claims 1-18 or the modified release formulation according to any of claims 19-35 and 45, wherein the first active agent and the second active agent are antigens derived from or corresponding to an infectious agent or a cancer.

52. A method of delivering therapeutic agents to a subject in need thereof, the method comprising administering the subject the pharmaceutical composition according to any of claims 1-18 or the modified release formulation according to any of claims 19-35 and 45, wherein the first active agent and the second active agent are therapeutic agents.

53. The method of claim 51 or 52, wherein the administering comprises intramuscular or subcutaneous injection of the pharmaceutical composition or modified release formulation.

54. The method of claim 51 or 52, wherein the administration of the pharmaceutical composition or modified release formulation is through transdermal or transmucosal route, such as oral or intranasal.
